(19) United States
(12) Patent Application Publication
(10) Pub. No.: US 2023/0301682 A1
(43) Pub. Date: Sep. 28, 2023

(54) CANNULA REDUCER

(71) Applicant: Auris Health, Inc., Redwood City, CA (US)

(72) Inventors: Andrew S. BERKOWITZ, Redwood City, CA (US); Travis Michael SCHUH, Los Altos, CA (US)

(21) Appl. No.: 18/324,810

(22) Filed: May 26, 2023

Related U.S. Application Data
(63) Continuation of application No. PCT/IB2021/061875, filed on Dec. 16, 2021.
(60) Provisional application No. 63/133,117, filed on Dec. 31, 2020.

(51) Int. Cl. A61B 17/34; A61B 34/20; A61B 90/00

(52) U.S. Cl. CPC A61B 17/3439; A61B 34/20; A61B 90/361; A61B 34/30

(57) ABSTRACT

A cannula reducer can include a sleeve having a proximal end and a distal end. The sleeve can be configured to be inserted into a cannula. The sleeve can have an interior passage configured to receive a surgical instrument. The proximal end of the sleeve is configured to attach to the surgical instrument.

### CROSS-REFERENCE TO RELATED APPLICATIONS

The present application is a continuation of International Patent Application No. PCT/IB2021/061875 filed Dec. 16, 2021 and entitled “CANNULA REDUCER,” which claims priority to U.S. Provisional Application No. 63/133,117 filed Dec. 31, 2020 and entitled, “CANNULA REDUCER,” both of which are incorporated herein by reference in their entirety for all purposes.

### TECHNICAL FIELD

Systems and methods disclosed herein are related to medical devices, and more particularly to cannula reducers.

### BACKGROUND

Minimally invasive procedures allow for access to a targeted site within a patient with minimal trauma to the patient. For example, laparoscopic surgery can allow for surgical access to a patient's cavity through a small incision on the patient's abdomen. A cannula can form a surgical corridor to allow elongate surgical instruments to access the internal anatomical site to perform a procedural function such as manipulating or visualizing tissue within the patient. During a surgery, the cannula can support and maintain the instrument in coaxial alignment with the cannula. In some applications, the cannula is sized or configured to support and/or align an instrument having a certain diameter.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 illustrates an embodiment of a cart-based robotic system arranged for diagnostic and/or therapeutic bronchoscopy procedures.

FIG. 2 depicts further aspects of the robotic system of FIG. 1.

FIG. 3 illustrates an embodiment of the robotic system of FIG. 1 arranged for ureteroscopy.

FIG. 4 illustrates an embodiment of the robotic system of FIG. 1 arranged for a vascular procedure.

FIG. 5 illustrates an embodiment of a table-based robotic system arranged for a bronchoscopy procedure.

FIG. 6 provides an alternative view of the robotic system of FIG. 5.

FIG. 7 illustrates an example system configured to stow robotic arm(s).

FIG. 8 illustrates an embodiment of a table-based robotic system configured for a ureteroscopy procedure.

FIG. 9 illustrates an embodiment of a table-based robotic system configured for a laparoscopic procedure.

FIG. 10 illustrates an embodiment of the table-based robotic system of FIGS. 5-9 with pitch or tilt adjustment.

FIG. 11 provides a detailed illustration of the interface between the table and the column of the table-based robotic system of FIGS. 5-10.

FIG. 12 illustrates an alternative embodiment of a table-based robotic system.

FIG. 13 illustrates an end view of the table-based robotic system of FIG. 12.

FIG. 14 illustrates an end view of a table-based robotic system with robotic arms attached thereto, according to some embodiments.

FIG. 15 illustrates an exemplary instrument driver.

FIG. 16 illustrates an exemplary surgical instrument with a paired instrument driver.

FIG. 17 illustrates an alternative design for an instrument driver and instrument where the axes of the drive units are parallel to the axis of the elongated shaft of the instrument, according to some embodiments.

FIG. 18 illustrates an instrument having an instrument-based insertion architecture, according to some embodiments.

FIG. 19 illustrates an exemplary controller.

FIG. 20 depicts a block diagram illustrating a localization system that estimates a location of one or more elements of the robotic systems of FIGS. 1-10, such as the location of the instrument of FIGS. 16-18, according to some embodiments.

FIG. 21 illustrates an exemplary docking arrangement of components of a robotic system, according to some embodiments.

FIG. 22 illustrates a cross-sectional view of the docking arrangement of FIG. 21, according to some embodiments.

FIG. 23 illustrates an exploded view of the docking arrangement of FIG. 21, according to some embodiments.

FIG. 24 illustrates a cannula reducer and surgical instrument, according to some embodiments.

FIG. 25 illustrates the cannula reducer and surgical instrument of FIG. 24 as an assembled unit, according to some embodiments.

FIG. 26 illustrates the cannula reducer and the surgical instrument docked with the robotic system, according to some embodiments.

FIG. 27 illustrates a latching mechanism of the cannula reducer, according to some embodiments.

FIG. 28 illustrates a cross-sectional view of the latching mechanism of FIG. 27, according to some embodiments.

FIG. 29 illustrates a distal end of the cannula reducer, according to some embodiments.

FIG. 30 illustrates the distal end of the cannula reducer within a cannula, according to some embodiments.

FIGS. 31-33 illustrate exemplary latching mechanisms for a cannula reducer, according to some embodiments.

FIG. 34 illustrates an exploded view of an exemplary cannula reducer and surgical instrument, according to some embodiments.

FIG. 35 illustrates the cannula reducer and surgical instrument of FIG. 34 as an assembled unit.

FIG. 36 illustrates an exploded view of an exemplary cannula reducer and surgical instrument, according to some embodiments.

FIG. 37 illustrates the cannula reducer and surgical instrument of FIG. 36 as a partially assembled unit.

FIG. 38 illustrates the cannula reducer and surgical instrument of FIG. 36 as an assembled unit.

FIG. 39 illustrates an exploded view of an exemplary cannula reducer and surgical instrument, according to some embodiments.

FIG. 40 illustrates the cannula reducer and surgical instrument of FIG. 39 as a partially assembled unit.

FIG. 41 illustrates the cannula reducer and surgical instrument of FIG. 39 as an assembled unit.

FIG. 42 illustrates an exploded view of a docking arrangement with an exemplary cannula reducer and an inserter, according to some embodiments.

FIG. 43 illustrates the cannula reducer and the inserter of FIG. 42 as an assembled unit.

FIG. 44 illustrates an exploded view of an exemplary cannula reducer and an inserter, according to some embodiments.

FIG. 45 illustrates the cannula reducer and the inserter of FIG. 44 as an assembled unit.

FIG. 46 illustrates an exploded view of an exemplary cannula reducer and an inserter, according to some embodiments.

FIG. 47 illustrates the cannula reducer and the inserter of FIG. 46 as an assembled unit.

FIGS. 48 and 49 illustrate flowcharts for methods of using a cannula reducer, according to some embodiments.

### DETAILED DESCRIPTION

### 1. Overview

Aspects of the present disclosure may be integrated into a robotically-enabled medical system capable of performing a variety of medical procedures, including both minimally invasive, such as laparoscopy, and non-invasive, such as endoscopy, procedures. Among endoscopy procedures, the system may be capable of performing bronchoscopy, ureteroscopy, gastroscopy, etc.

In addition to performing the breadth of procedures, the system may provide additional benefits, such as enhanced imaging and guidance to assist the physician. Additionally, the system may provide the physician with the ability to perform the procedure from an ergonomic position without the need for awkward arm motions and positions. Still further, the system may provide the physician with the ability to perform the procedure with improved ease of use such that one or more of the instruments of the system can be controlled by a single user.

Various embodiments will be described below in conjunction with the drawings for purposes of illustration. It should be appreciated that many other implementations of the disclosed concepts are possible, and various advantages can be achieved with the disclosed implementations. Headings are included herein for reference and to aid in locating various sections. These headings are not intended to limit the scope of the concepts described with respect thereto. Such concepts may have applicability throughout the entire specification.

### A. Robotic System—Cart.

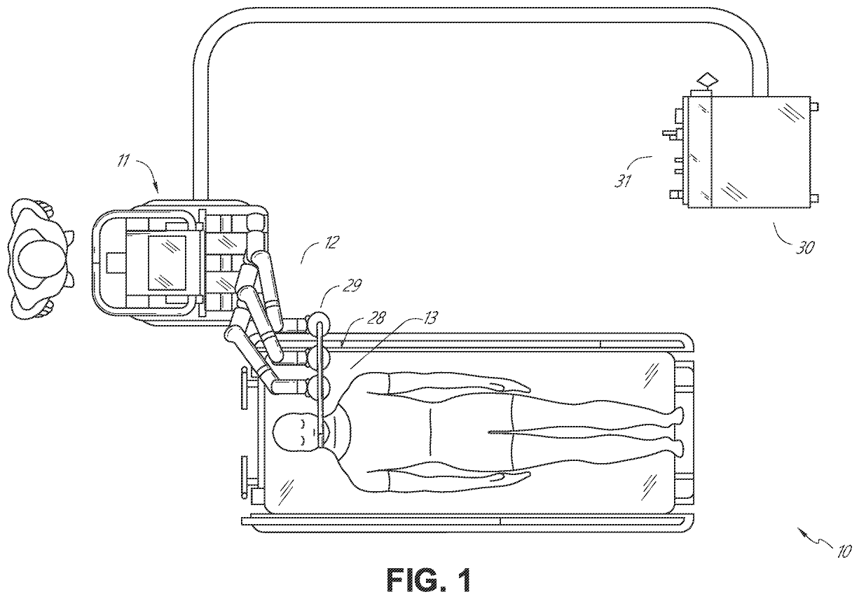

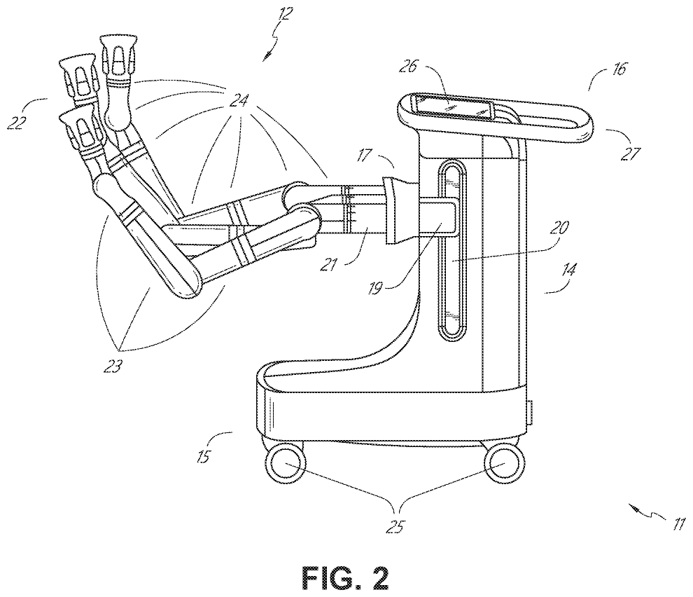

The robotically-enabled medical system may be configured in a variety of ways depending on the particular procedure. FIG. 1 illustrates an embodiment of a cart-based robotically-enabled system 10 arranged for a diagnostic and/or therapeutic bronchoscopy procedure. During a bronchoscopy, the system 10 may comprise a cart 11 having one or more robotic arms 12 to deliver a medical instrument, such as a steerable endoscope 13, which may be a procedure-specific bronchoscope for bronchoscopy, to a natural orifice access point (i.e., the mouth of the patient positioned on a table in the present example) to deliver diagnostic and/or therapeutic tools. As shown, the cart 11 may be positioned proximate to the patient's upper torso in order to provide access to the access point. Similarly, the robotic arms 12 may be actuated to position the bronchoscope relative to the access point. The arrangement in FIG. 1 may also be utilized when performing a gastro-intestinal (GI) procedure with a gastroscope, a specialized endoscope for GI procedures. FIG. 2 depicts an example embodiment of the cart in greater detail.

With continued reference to FIG. 1, once the cart 11 is properly positioned, the robotic arms 12 may insert the steerable endoscope 13 into the patient robotically, manually, or a combination thereof. As shown, the steerable endoscope 13 may comprise at least two telescoping parts, such as an inner leader portion and an outer sheath portion, each portion coupled to a separate instrument driver from the set of instrument drivers 28, each instrument driver coupled to the distal end of an individual robotic arm. This linear arrangement of the instrument drivers 28, which facilitates coaxially aligning the leader portion with the sheath portion, creates a “virtual rail” 29 that may be repositioned in space by manipulating the one or more robotic arms 12 into different angles and/or positions. The virtual rails described herein are depicted in the Figures using dashed lines, and accordingly the dashed lines do not depict any physical structure of the system. Translation of the instrument drivers 28 along the virtual rail 29 telescopes the inner leader portion relative to the outer sheath portion or advances or retracts the endoscope 13 from the patient. The angle of the virtual rail 29 may be adjusted, translated, and pivoted based on clinical application or physician preference. For example, in bronchoscopy, the angle and position of the virtual rail 29 as shown represents a compromise between providing physician access to the endoscope 13 while minimizing friction that results from bending the endoscope 13 into the patient's mouth.

The endoscope 13 may be directed down the patient's trachea and lungs after insertion using precise commands from the robotic system until reaching the target destination or operative site. In order to enhance navigation through the patient's lung network and/or reach the desired target, the endoscope 13 may be manipulated to telescopically extend the inner leader portion from the outer sheath portion to obtain enhanced articulation and greater bend radius. The use of separate instrument drivers 28 also allows the leader portion and sheath portion to be driven independent of each other.

For example, the endoscope 13 may be directed to deliver a biopsy needle to a target, such as, for example, a lesion or nodule within the lungs of a patient. The needle may be deployed down a working channel that runs the length of the endoscope to obtain a tissue sample to be analyzed by a pathologist. Depending on the pathology results, additional tools may be deployed down the working channel of the endoscope for additional biopsies. After identifying a nodule to be malignant, the endoscope 13 may endoscopically deliver tools to resect the potentially cancerous tissue. In some instances, diagnostic and therapeutic treatments can be delivered in separate procedures. In those circumstances, the endoscope 13 may also be used to deliver a fiducial to “mark” the location of the target nodule as well. In other instances, diagnostic and therapeutic treatments may be delivered during the same procedure.

The system 10 may also include a movable tower 30, which may be connected via support cables to the cart 11 to provide support for controls, electronics, fluidics, optics, sensors, and/or power to the cart 11. Placing such functionality in the tower 30 allows for a smaller form factor cart 11 that may be more easily adjusted and/or re-positioned by an operating physician and his/her staff. Additionally, the division of functionality between the cart/table and the support tower 30 reduces operating room clutter and facilitates improving clinical workflow. While the cart 11 may be positioned close to the patient, the tower 30 may be stowed in a remote location to stay out of the way during a procedure.

In support of the robotic systems described above, the tower 30 may include component(s) of a computer-based control system that stores computer program instructions, for example, within a non-transitory computer-readable storage medium such as a persistent magnetic storage drive, solid state drive, etc. The execution of those instructions, whether the execution occurs in the tower 30 or the cart 11, may control the entire system or sub-system(s) thereof. For example, when executed by a processor of the computer system, the instructions may cause the components of the robotics system to actuate the relevant carriages and arm mounts, actuate the robotics arms, and control the medical instruments. For example, in response to receiving the control signal, the motors in the joints of the robotics arms may position the arms into a certain posture.

The tower 30 may also include a pump, flow meter, valve control, and/or fluid access in order to provide controlled irrigation and aspiration capabilities to the system that may be deployed through the endoscope 13. These components may also be controlled using the computer system of tower 30. In some embodiments, irrigation and aspiration capabilities may be delivered directly to the endoscope 13 through separate cable(s).

The tower 30 may include a voltage and surge protector designed to provide filtered and protected electrical power to the cart 11, thereby avoiding placement of a power transformer and other auxiliary power components in the cart 11, resulting in a smaller, more moveable cart 11.

The tower 30 may also include support equipment for the sensors deployed throughout the robotic system 10. For example, the tower 30 may include opto-electronics equipment for detecting, receiving, and processing data received from the optical sensors or cameras throughout the robotic system 10. In combination with the control system, such opto-electronics equipment may be used to generate real-time images for display in any number of consoles deployed throughout the system, including in the tower 30. Similarly, the tower 30 may also include an electronic subsystem for receiving and processing signals received from deployed electromagnetic (EM) sensors. The tower 30 may also be used to house and position an EM field generator for detection by EM sensors in or on the medical instrument.

The tower 30 may also include a console 31 in addition to other consoles available in the rest of the system, e.g., console mounted on top of the cart. The console 31 may include a user interface and a display screen, such as a touchscreen, for the physician operator. Consoles in system 10 are generally designed to provide both robotic controls as well as pre-operative and real-time information of the procedure, such as navigational and localization information of the endoscope 13. When the console 31 is not the only console available to the physician, it may be used by a second operator, such as a nurse, to monitor the health or vitals of the patient and the operation of system, as well as provide procedure-specific data, such as navigational and localization information. In other embodiments, the console 30 is housed in a body that is separate from the tower 30.

The tower 30 may be coupled to the cart 11 and endoscope 13 through one or more cables or connections (not shown). In some embodiments, the support functionality from the tower 30 may be provided through a single cable to the cart 11, simplifying and de-cluttering the operating room. In other embodiments, specific functionality may be coupled in separate cabling and connections. For example, while power may be provided through a single power cable to the cart, the support for controls, optics, fluidics, and/or navigation may be provided through a separate cable.

FIG. 2 provides a detailed illustration of an embodiment of the cart from the cart-based robotically-enabled system shown in FIG. 1. The cart 11 generally includes an elongated support structure 14 (often referred to as a “column”), a cart base 15, and a console 16 at the top of the column 14. The column 14 may include one or more carriages, such as a carriage 17 (alternatively “arm support”) for supporting the deployment of one or more robotic arms 12 (three shown in FIG. 2). The carriage 17 may include individually configurable arm mounts that rotate along a perpendicular axis to adjust the base of the robotic arms 12 for better positioning relative to the patient. The carriage 17 also includes a carriage interface 19 that allows the carriage 17 to vertically translate along the column 14.

The carriage interface 19 is connected to the column 14 through slots, such as slot 20, that are positioned on opposite sides of the column 14 to guide the vertical translation of the carriage 17. The slot 20 contains a vertical translation interface to position and hold the carriage at various vertical heights relative to the cart base 15. Vertical translation of the carriage 17 allows the cart 11 to adjust the reach of the robotic arms 12 to meet a variety of table heights, patient sizes, and physician preferences. Similarly, the individually configurable arm mounts on the carriage 17 allow the robotic arm base 21 of robotic arms 12 to be angled in a variety of configurations.

In some embodiments, the slot 20 may be supplemented with slot covers that are flush and parallel to the slot surface to prevent dirt and fluid ingress into the internal chambers of the column 14 and the vertical translation interface as the carriage 17 vertically translates. The slot covers may be deployed through pairs of spring spools positioned near the vertical top and bottom of the slot 20. The covers are coiled within the spools until deployed to extend and retract from their coiled state as the carriage 17 vertically translates up and down. The spring-loading of the spools provides force to retract the cover into a spool when carriage 17 translates towards the spool, while also maintaining a tight seal when the carriage 17 translates away from the spool. The covers may be connected to the carriage 17 using, for example, brackets in the carriage interface 19 to ensure proper extension and retraction of the cover as the carriage 17 translates.

The column 14 may internally comprise mechanisms, such as gears and motors, that are designed to use a vertically aligned lead screw to translate the carriage 17 in a mechanized fashion in response to control signals generated in response to user inputs, e.g., inputs from the console 16.

The robotic arms 12 may generally comprise robotic arm bases 21 and end effectors 22, separated by a series of linkages 23 that are connected by a series of joints 24, each joint comprising an independent actuator, each actuator comprising an independently controllable motor. Each independently controllable joint represents an independent degree of freedom available to the robotic arm. Each of the arms 12 have seven joints, and thus provide seven degrees of freedom. A multitude of joints result in a multitude of degrees of freedom, allowing for “redundant” degrees of freedom. Redundant degrees of freedom allow the robotic arms 12 to position their respective end effectors 22 at a specific position, orientation, and trajectory in space using different linkage positions and joint angles. This allows for the system to position and direct a medical instrument from a desired point in space while allowing the physician to move the arm joints into a clinically advantageous position away from the patient to create greater access, while avoiding arm collisions.

The cart base 15 balances the weight of the column 14, carriage 17, and arms 12 over the floor. Accordingly, the cart base 15 houses heavier components, such as electronics, motors, power supply, as well as components that either enable movement and/or immobilize the cart. For example, the cart base 15 includes rollable wheel-shaped casters 25 that allow for the cart to easily move around the room prior to a procedure. After reaching the appropriate position, the casters 25 may be immobilized using wheel locks to hold the cart 11 in place during the procedure.

Positioned at the vertical end of column 14, the console 16 allows for both a user interface for receiving user input and a display screen (or a dual-purpose device such as, for example, a touchscreen 26) to provide the physician user with both pre-operative and intra-operative data. Potential pre-operative data on the touchscreen 26 may include pre-operative plans, navigation and mapping data derived from pre-operative computerized tomography (CT) scans, and/or notes from pre-operative patient interviews. Intra-operative data on display may include optical information provided from the tool, sensor and coordinate information from sensors, as well as vital patient statistics, such as respiration, heart rate, and/or pulse. The console 16 may be positioned and tilted to allow a physician to access the console from the side of the column 14 opposite carriage 17. From this position, the physician may view the console 16, robotic arms 12, and patient while operating the console 16 from behind the cart 11. As shown, the console 16 also includes a handle 27 to assist with maneuvering and stabilizing cart 11.

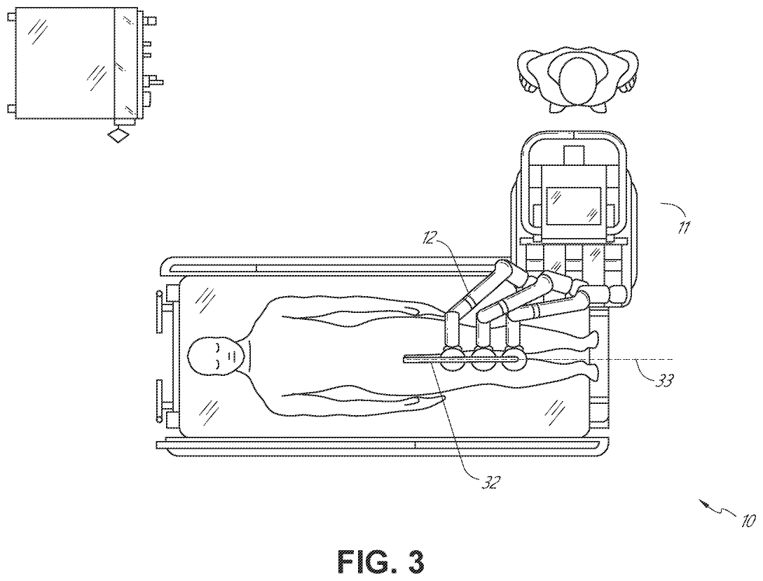

FIG. 3 illustrates an embodiment of a robotically-enabled system 10 arranged for ureteroscopy. In a ureteroscopic procedure, the cart 11 may be positioned to deliver a ureteroscope 32, a procedure-specific endoscope designed to traverse a patient's urethra and ureter, to the lower abdominal area of the patient. In a ureteroscopy, it may be desirable for the ureteroscope 32 to be directly aligned with the patient's urethra to reduce friction and forces on the sensitive anatomy in the area. As shown, the cart 11 may be aligned at the foot of the table to allow the robotic arms 12 to position the ureteroscope 32 for direct linear access to the patient's urethra. From the foot of the table, the robotic arms 12 may insert the ureteroscope 32 along the virtual rail 33 directly into the patient's lower abdomen through the urethra.

After insertion into the urethra, using similar control techniques as in bronchoscopy, the ureteroscope 32 may be navigated into the bladder, ureters, and/or kidneys for diagnostic and/or therapeutic applications. For example, the ureteroscope 32 may be directed into the ureter and kidneys to break up kidney stone build up using a laser or ultrasonic lithotripsy device deployed down the working channel of the ureteroscope 32. After lithotripsy is complete, the resulting stone fragments may be removed using baskets deployed down the ureteroscope 32.

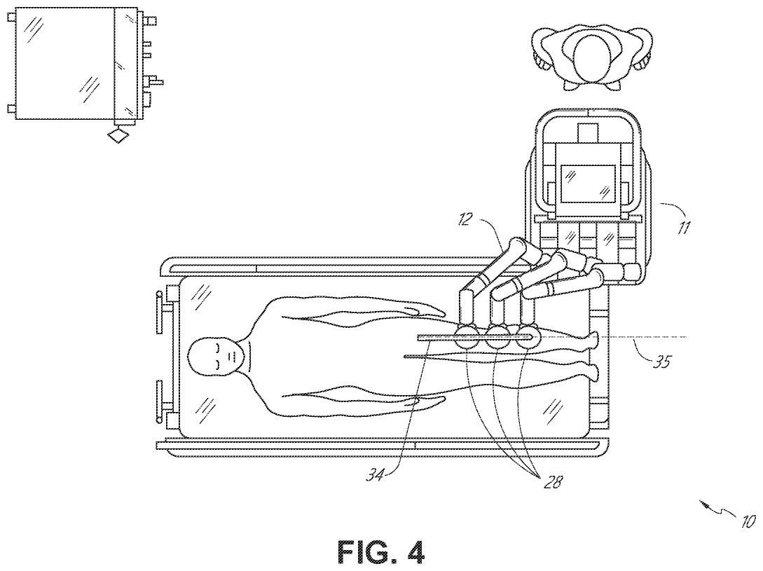

FIG. 4 illustrates an embodiment of a robotically-enabled system similarly arranged for a vascular procedure. In a vascular procedure, the system 10 may be configured such that the cart 11 may deliver a medical instrument 34, such as a steerable catheter, to an access point in the femoral artery in the patient's leg. The femoral artery presents both a larger diameter for navigation as well as a relatively less circuitous and tortuous path to the patient's heart, which simplifies navigation. As in a ureteroscopic procedure, the cart 11 may be positioned towards the patient's legs and lower abdomen to allow the robotic arms 12 to provide a virtual rail 35 with direct linear access to the femoral artery access point in the patient's thigh/hip region. After insertion into the artery, the medical instrument 34 may be directed and inserted by translating the instrument drivers 28. Alternatively, the cart may be positioned around the patient's upper abdomen in order to reach alternative vascular access points, such as, for example, the carotid and brachial arteries near the shoulder and wrist.

### B. Robotic System—Table.

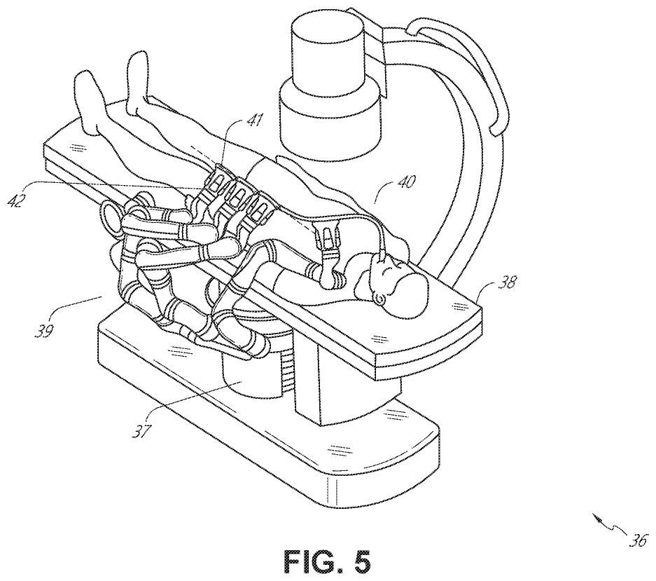

Embodiments of the robotically-enabled medical system may also incorporate the patient's table. Incorporation of the table reduces the amount of capital equipment within the operating room by removing the cart, which allows greater access to the patient. FIG. 5 illustrates an embodiment of such a robotically-enabled system arranged for a bronchoscopy procedure. System 36 includes a support structure or column 37 for supporting platform 38 (shown as a “table” or “bed”) over the floor. Much like in the cart-based systems, the end effectors of the robotic arms 39 of the system 36 comprise instrument drivers 42 that are designed to manipulate an elongated medical instrument, such as a bronchoscope 40 in FIG. 5, through or along a virtual rail 41 formed from the linear alignment of the instrument drivers 42. In practice, a C-arm for providing fluoroscopic imaging may be positioned over the patient's upper abdominal area by placing the emitter and detector around table 38.

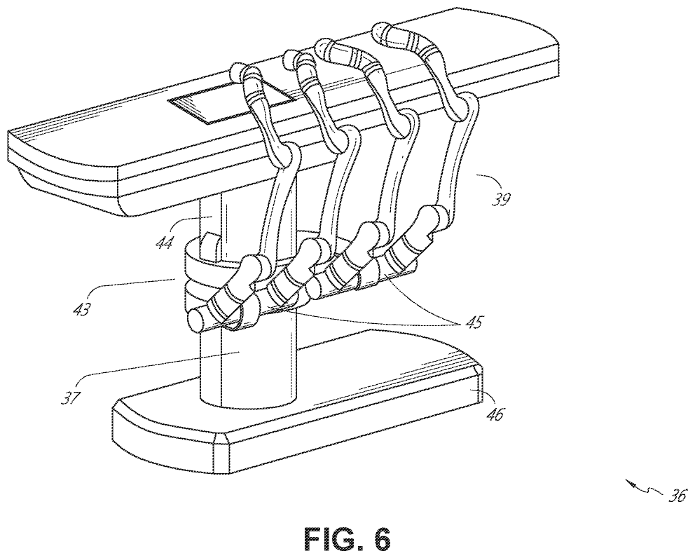

FIG. 6 provides an alternative view of the system 36 without the patient and medical instrument for discussion purposes. As shown, the column 37 may include one or more carriages 43 shown as ring-shaped in the system 36, from which the one or more robotic arms 39 may be based. The carriages 43 may translate along a vertical column interface 44 that runs the length of the column 37 to provide different vantage points from which the robotic arms 39 may be positioned to reach the patient. The carriage(s) 43 may rotate around the column 37 using a mechanical motor positioned within the column 37 to allow the robotic arms 39 to have access to multiples sides of the table 38, such as, for example, both sides of the patient. In embodiments with multiple carriages, the carriages may be individually positioned on the column and may translate and/or rotate independent of the other carriages. While carriages 43 need not surround the column 37 or even be circular, the ring-shape as shown facilitates rotation of the carriages 43 around the column 37 while maintaining structural balance. Rotation and translation of the carriages 43 allows the system to align the medical instruments, such as endoscopes and laparoscopes, into different access points on the patient. In other embodiments (not shown), the system 36 can include a patient table or bed with adjustable arm supports in the form of bars or rails extending alongside it. One or more robotic arms 39 (e.g., via a shoulder with an elbow joint) can be attached to the adjustable arm supports, which can be vertically adjusted. By providing vertical adjustment, the robotic arms 39 are advantageously capable of being stowed compactly beneath the patient table or bed, and subsequently raised during a procedure.

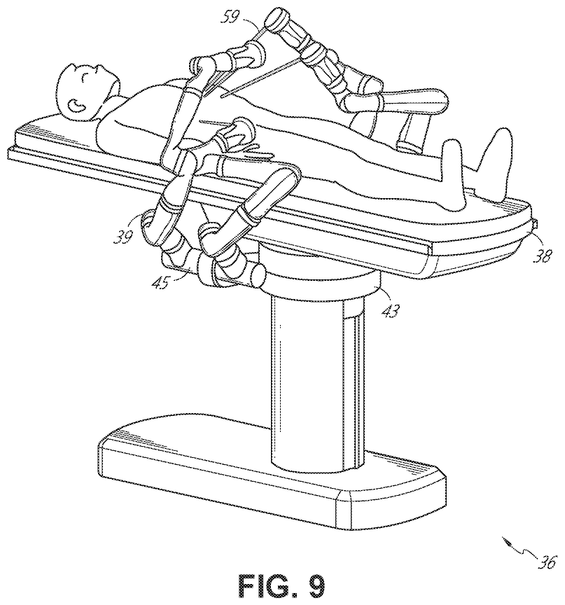

The arms 39 may be mounted on the carriages through a set of arm mounts 45 comprising a series of joints that may individually rotate and/or telescopically extend to provide additional configurability to the robotic arms 39. Additionally, the arm mounts 45 may be positioned on the carriages 43 such that, when the carriages 43 are appropriately rotated, the arm mounts 45 may be positioned on either the same side of table 38 (as shown in FIG. 6), on opposite sides of table 38 (as shown in FIG. 9), or on adjacent sides of the table 38 (not shown).

The column 37 structurally provides support for the table 38, and a path for vertical translation of the carriages. Internally, the column 37 may be equipped with lead screws for guiding vertical translation of the carriages, and motors to mechanize the translation of said carriages based the lead screws. The column 37 may also convey power and control signals to the carriage 43 and robotic arms 39 mounted thereon.

The table base 46 serves a similar function as the cart base 15 in cart 11 shown in FIG. 2, housing heavier components to balance the table/bed 38, the column 37, the carriages 43, and the robotic arms 39. The table base 46 may also incorporate rigid casters to provide stability during procedures. Deployed from the bottom of the table base 46, the casters may extend in opposite directions on both sides of the base 46 and retract when the system 36 needs to be moved.

Continuing with FIG. 6, the system 36 may also include a tower (not shown) that divides the functionality of system 36 between table and tower to reduce the form factor and bulk of the table. As in earlier disclosed embodiments, the tower may provide a variety of support functionalities to table, such as processing, computing, and control capabilities, power, fluidics, and/or optical and sensor processing. The tower may also be movable to be positioned away from the patient to improve physician access and de-clutter the operating room. Additionally, placing components in the tower allows for more storage space in the table base for potential stowage of the robotic arms. The tower may also include a master controller or console that provides both a user interface for user input, such as keyboard and/or pendant, as well as a display screen (or touchscreen) for pre-operative and intra-operative information, such as real-time imaging, navigation, and tracking information. In some embodiments, the tower may also contain holders for gas tanks to be used for insufflation.

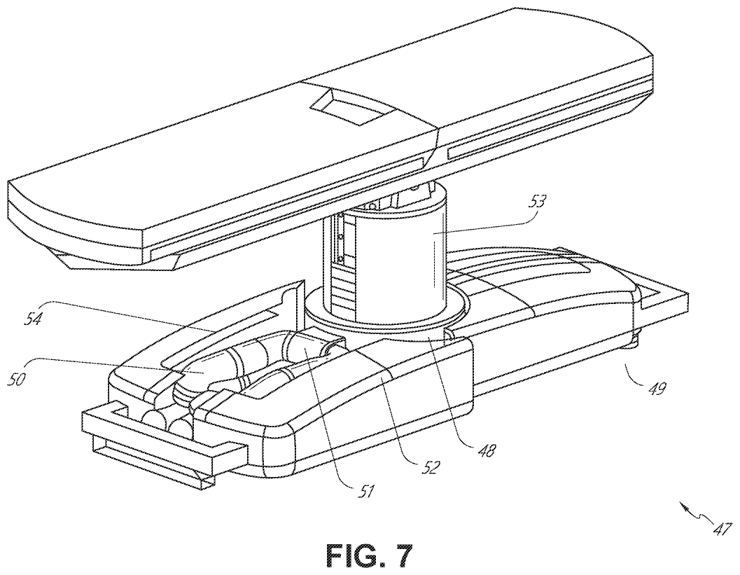

In some embodiments, a table base may stow and store the robotic arms when not in use. FIG. 7 illustrates a system 47 that stows robotic arms in an embodiment of the table-based system. In system 47, carriages 48 may be vertically translated into base 49 to stow robotic arms 50, arm mounts 51, and the carriages 48 within the base 49. Base covers 52 may be translated and retracted open to deploy the carriages 48, arm mounts 51, and arms 50 around column 53, and closed to stow to protect them when not in use. The base covers 52 may be sealed with a membrane 54 along the edges of its opening to prevent dirt and fluid ingress when closed.

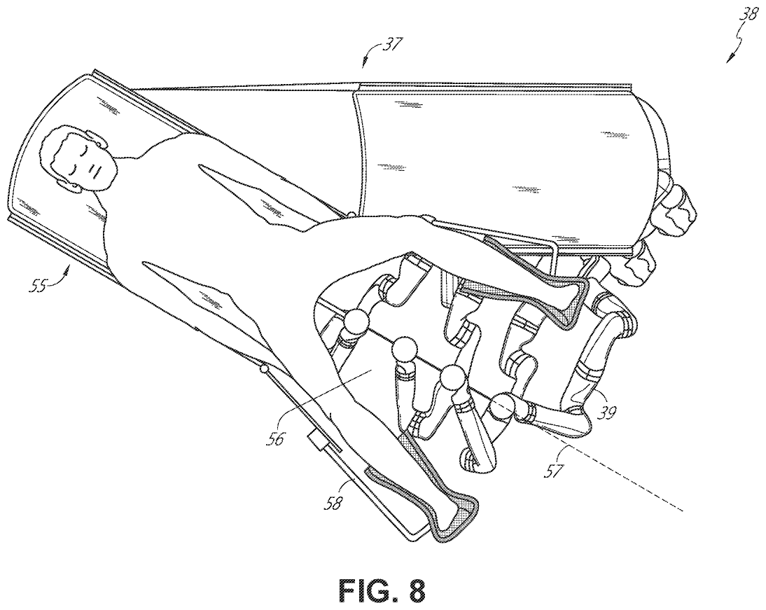

FIG. 8 illustrates an embodiment of a robotically-enabled table-based system configured for a ureteroscopy procedure. In a ureteroscopy, the table 38 may include a swivel portion 55 for positioning a patient off-angle from the column 37 and table base 46. The swivel portion 55 may rotate or pivot around a pivot point (e.g., located below the patient's head) in order to position the bottom portion of the swivel portion 55 away from the column 37. For example, the pivoting of the swivel portion 55 allows a C-arm (not shown) to be positioned over the patient's lower abdomen without competing for space with the column (not shown) below table 38. By rotating the carriage (not shown) around the column 37, the robotic arms 39 may directly insert a ureteroscope 56 along a virtual rail 57 into the patient's groin area to reach the urethra. In a ureteroscopy, stirrups 58 may also be fixed to the swivel portion 55 of the table 38 to support the position of the patient's legs during the procedure and allow clear access to the patient's groin area.

In a laparoscopic procedure, through small incision(s) in the patient's abdominal wall, minimally invasive instruments may be inserted into the patient's anatomy. In some embodiments, the minimally invasive instruments comprise an elongated rigid member, such as a shaft, which is used to access anatomy within the patient. After inflation of the patient's abdominal cavity, the instruments may be directed to perform surgical or medical tasks, such as grasping, cutting, ablating, suturing, etc. In some embodiments, the instruments can comprise a scope, such as a laparoscope. FIG. 9 illustrates an embodiment of a robotically-enabled table-based system configured for a laparoscopic procedure. As shown in FIG. 9, the carriages 43 of the system 36 may be rotated and vertically adjusted to position pairs of the robotic arms 39 on opposite sides of the table 38, such that instrument 59 may be positioned using the arm mounts 45 to be passed through minimal incisions on both sides of the patient to reach his/her abdominal cavity.

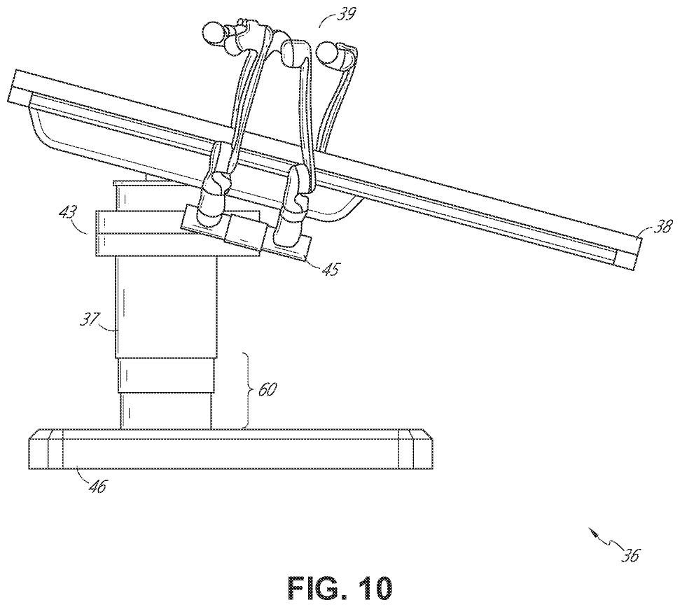

To accommodate laparoscopic procedures, the robotically-enabled table system may also tilt the platform to a desired angle. FIG. 10 illustrates an embodiment of the robotically-enabled medical system with pitch or tilt adjustment. As shown in FIG. 10, the system 36 may accommodate tilt of the table 38 to position one portion of the table at a greater distance from the floor than the other. Additionally, the arm mounts 45 may rotate to match the tilt such that the arms 39 maintain the same planar relationship with table 38. To accommodate steeper angles, the column 37 may also include telescoping portions 60 that allow vertical extension of column 37 to keep the table 38 from touching the floor or colliding with base 46.

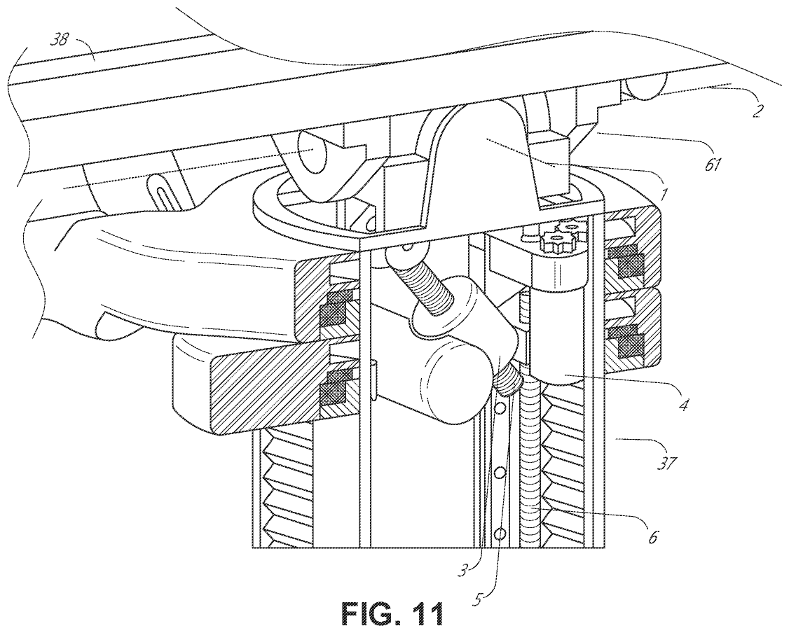

FIG. 11 provides a detailed illustration of the interface between the table 38 and the column 37. Pitch rotation mechanism 61 may be configured to alter the pitch angle of the table 38 relative to the column 37 in multiple degrees of freedom. The pitch rotation mechanism 61 may be enabled by the positioning of orthogonal axes 1, 2 at the column-table interface, each axis actuated by a separate motor 3, 4 responsive to an electrical pitch angle command. Rotation along one screw 5 would enable tilt adjustments in one axis 1, while rotation along the other screw 6 would enable tilt adjustments along the other axis 2. In some embodiments, a ball joint can be used to alter the pitch angle of the table 38 relative to the column 37 in multiple degrees of freedom.

For example, pitch adjustments are particularly useful when trying to position the table in a Trendelenburg position, i.e., position the patient's lower abdomen at a higher position from the floor than the patient's lower abdomen, for lower abdominal surgery. The Trendelenburg position causes the patient's internal organs to slide towards his/her upper abdomen through the force of gravity, clearing out the abdominal cavity for minimally invasive tools to enter and perform lower abdominal surgical or medical procedures, such as laparoscopic prostatectomy.

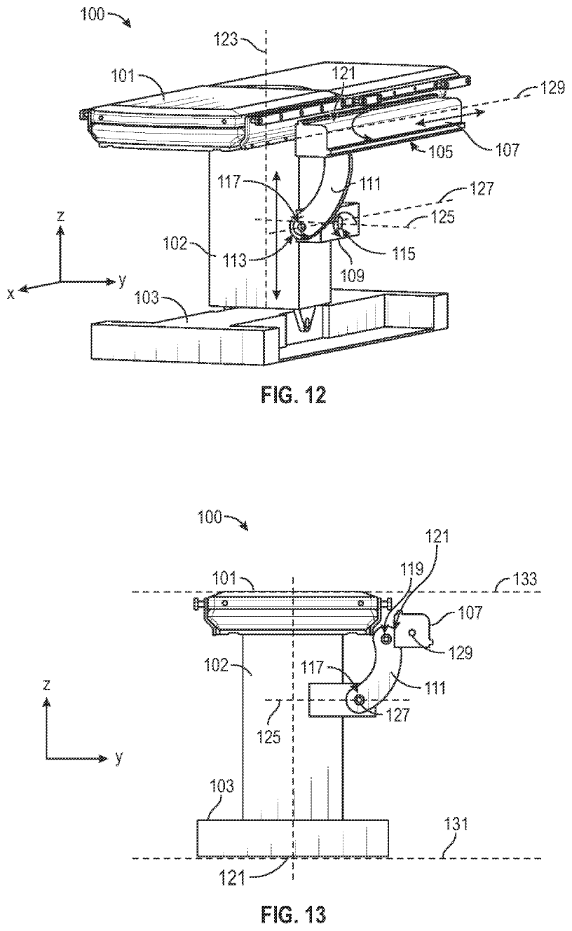

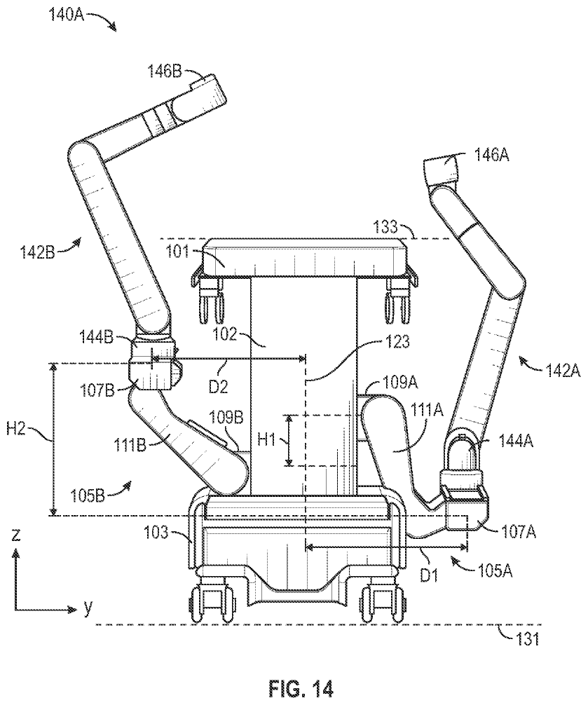

FIGS. 12 and 13 illustrate isometric and end views of an alternative embodiment of a table-based surgical robotics system 100. The surgical robotics system 100 includes one or more adjustable arm supports 105 that can be configured to support one or more robotic arms (see, for example, FIG. 14) relative to a table 101. In the illustrated embodiment, a single adjustable arm support 105 is shown, though an additional arm support can be provided on an opposite side of the table 101. The adjustable arm support 105 can be configured so that it can move relative to the table 101 to adjust and/or vary the position of the adjustable arm support 105 and/or any robotic arms mounted thereto relative to the table 101. For example, the adjustable arm support 105 may be adjusted one or more degrees of freedom relative to the table 101. The adjustable arm support 105 provides high versatility to the system 100, including the ability to easily stow the one or more adjustable arm supports 105 and any robotics arms attached thereto beneath the table 101. The adjustable arm support 105 can be elevated from the stowed position to a position below an upper surface of the table 101. In other embodiments, the adjustable arm support 105 can be elevated from the stowed position to a position above an upper surface of the table 101.

The adjustable arm support 105 can provide several degrees of freedom, including lift, lateral translation, tilt, etc. In the illustrated embodiment of FIGS. 12 and 13, the arm support 105 is configured with four degrees of freedom, which are illustrated with arrows in FIG. 12. A first degree of freedom allows for adjustment of the adjustable arm support 105 in the z-direction (“Z-lift”). For example, the adjustable arm support 105 can include a carriage 109 configured to move up or down along or relative to a column 102 supporting the table 101. A second degree of freedom can allow the adjustable arm support 105 to tilt. For example, the adjustable arm support 105 can include a rotary joint, which can allow the adjustable arm support 105 to be aligned with the bed in a Trendelenburg position. A third degree of freedom can allow the adjustable arm support 105 to “pivot up,” which can be used to adjust a distance between a side of the table 101 and the adjustable arm support 105. A fourth degree of freedom can permit translation of the adjustable arm support 105 along a longitudinal length of the table.

The surgical robotics system 100 in FIGS. 12 and 13 can comprise a table supported by a column 102 that is mounted to a base 103. The base 103 and the column 102 support the table 101 relative to a support surface. A floor axis 131 and a support axis 133 are shown in FIG. 13.

The adjustable arm support 105 can be mounted to the column 102. In other embodiments, the arm support 105 can be mounted to the table 101 or base 103. The adjustable arm support 105 can include a carriage 109, a bar or rail connector 111 and a bar or rail 107. In some embodiments, one or more robotic arms mounted to the rail 107 can translate and move relative to one another.

The carriage 109 can be attached to the column 102 by a first joint 113, which allows the carriage 109 to move relative to the column 102 (e.g., such as up and down a first or vertical axis 123). The first joint 113 can provide the first degree of freedom (“Z-lift”) to the adjustable arm support 105. The adjustable arm support 105 can include a second joint 115, which provides the second degree of freedom (tilt) for the adjustable arm support 105. The adjustable arm support 105 can include a third joint 117, which can provide the third degree of freedom (“pivot up”) for the adjustable arm support 105. An additional joint 119 (shown in FIG. 13) can be provided that mechanically constrains the third joint 117 to maintain an orientation of the rail 107 as the rail connector 111 is rotated about a third axis 127. The adjustable arm support 105 can include a fourth joint 121, which can provide a fourth degree of freedom (translation) for the adjustable arm support 105 along a fourth axis 129.

FIG. 14 illustrates an end view of the surgical robotics system 140A with two adjustable arm supports 105A, 105B mounted on opposite sides of a table 101. A first robotic arm 142A is attached to the bar or rail 107A of the first adjustable arm support 105B. The first robotic arm 142A includes a base 144A attached to the rail 107A. The distal end of the first robotic arm 142A includes an instrument drive mechanism 146A that can attach to one or more robotic medical instruments or tools. Similarly, the second robotic arm 142B includes a base 144B attached to the rail 107B. The distal end of the second robotic arm 142B includes an instrument drive mechanism 146B. The instrument drive mechanism 146B can be configured to attach to one or more robotic medical instruments or tools.

In some embodiments, one or more of the robotic arms 142A, 142B comprises an arm with seven or more degrees of freedom. In some embodiments, one or more of the robotic arms 142A, 142B can include eight degrees of freedom, including an insertion axis (1-degree of freedom including insertion), a wrist (3-degrees of freedom including wrist pitch, yaw and roll), an elbow (1-degree of freedom including elbow pitch), a shoulder (2-degrees of freedom including shoulder pitch and yaw), and base 144A, 144B (1-degree of freedom including translation). In some embodiments, the insertion degree of freedom can be provided by the robotic arm 142A, 142B, while in other embodiments, the instrument itself provides insertion via an instrument-based insertion architecture.

### C. Instrument Driver & Interface.

The end effectors of the system's robotic arms comprise (i) an instrument driver (alternatively referred to as “instrument drive mechanism” or “instrument device manipulator”) that incorporate electro-mechanical means for actuating the medical instrument and (ii) a removable or detachable medical instrument, which may be devoid of any electro-mechanical components, such as motors. This dichotomy may be driven by the need to sterilize medical instruments used in medical procedures, and the inability to adequately sterilize expensive capital equipment due to their intricate mechanical assemblies and sensitive electronics. Accordingly, the medical instruments may be designed to be detached, removed, and interchanged from the instrument driver (and thus the system) for individual sterilization or disposal by the physician or the physician's staff. In contrast, the instrument drivers need not be changed or sterilized, and may be draped for protection.

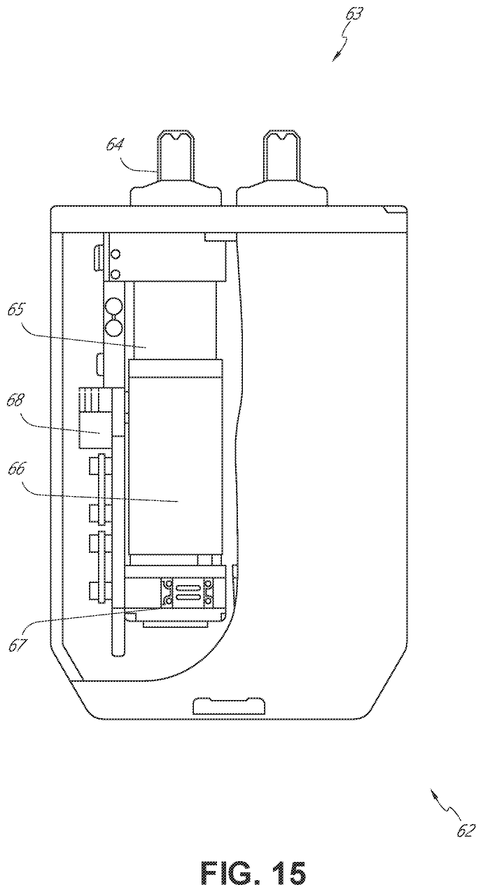

FIG. 15 illustrates an example instrument driver. Positioned at the distal end of a robotic arm, instrument driver 62 comprises of one or more drive units 63 arranged with parallel axes to provide controlled torque to a medical instrument via drive shafts 64. Each drive unit 63 comprises an individual drive shaft 64 for interacting with the instrument, a gear head 65 for converting the motor shaft rotation to a desired torque, a motor 66 for generating the drive torque, an encoder 67 to measure the speed of the motor shaft and provide feedback to the control circuitry, and control circuitry 68 for receiving control signals and actuating the drive unit. Each drive unit 63 being independent controlled and motorized, the instrument driver 62 may provide multiple (four as shown in FIG. 15) independent drive outputs to the medical instrument. In operation, the control circuitry 68 would receive a control signal, transmit a motor signal to the motor 66, compare the resulting motor speed as measured by the encoder 67 with the desired speed, and modulate the motor signal to generate the desired torque.

For procedures that require a sterile environment, the robotic system may incorporate a drive interface, such as a sterile adapter connected to a sterile drape, that sits between the instrument driver and the medical instrument. The chief purpose of the sterile adapter is to transfer angular motion from the drive shafts of the instrument driver to the drive inputs of the instrument while maintaining physical separation, and thus sterility, between the drive shafts and drive inputs. Accordingly, an example sterile adapter may comprise of a series of rotational inputs and outputs intended to be mated with the drive shafts of the instrument driver and drive inputs on the instrument. Connected to the sterile adapter, the sterile drape, comprised of a thin, flexible material such as transparent or translucent plastic, is designed to cover the capital equipment, such as the instrument driver, robotic arm, and cart (in a cart-based system) or table (in a table-based system). Use of the drape would allow the capital equipment to be positioned proximate to the patient while still being located in an area not requiring sterilization (i.e., non-sterile field). On the other side of the sterile drape, the medical instrument may interface with the patient in an area requiring sterilization (i.e., sterile field).

### D. Medical Instrument.

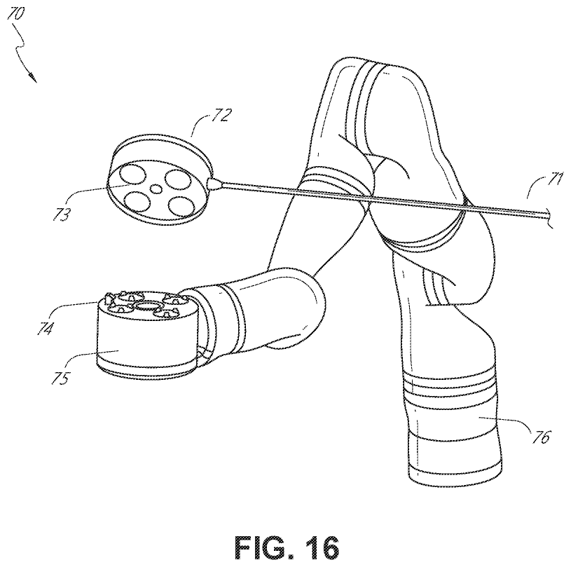

FIG. 16 illustrates an example medical instrument with a paired instrument driver. Like other instruments designed for use with a robotic system, medical instrument 70 comprises an elongated shaft 71 (or elongate body) and an instrument base 72. The instrument base 72, also referred to as an “instrument handle” due to its intended design for manual interaction by the physician, may generally comprise rotatable drive inputs 73, e.g., receptacles, pulleys or spools, that are designed to be mated with drive outputs 74 that extend through a drive interface on instrument driver 75 at the distal end of robotic arm 76. When physically connected, latched, and/or coupled, the mated drive inputs 73 of instrument base 72 may share axes of rotation with the drive outputs 74 in the instrument driver 75 to allow the transfer of torque from drive outputs 74 to drive inputs 73. In some embodiments, the drive outputs 74 may comprise splines that are designed to mate with receptacles on the drive inputs 73.

The elongated shaft 71 is designed to be delivered through either an anatomical opening or lumen, e.g., as in endoscopy, or a minimally invasive incision, e.g., as in laparoscopy. The elongated shaft 71 may be either flexible (e.g., having properties similar to an endoscope) or rigid (e.g., having properties similar to a laparoscope) or contain a customized combination of both flexible and rigid portions. When designed for laparoscopy, the distal end of a rigid elongated shaft may be connected to an end effector extending from a jointed wrist formed from a clevis with at least one degree of freedom and a surgical tool or medical instrument, such as, for example, a grasper or scissors, that may be actuated based on force from the tendons as the drive inputs rotate in response to torque received from the drive outputs 74 of the instrument driver 75. When designed for endoscopy, the distal end of a flexible elongated shaft may include a steerable or controllable bending section that may be articulated and bent based on torque received from the drive outputs 74 of the instrument driver 75.

Torque from the instrument driver 75 is transmitted down the elongated shaft 71 using tendons along the shaft 71. These individual tendons, such as pull wires, may be individually anchored to individual drive inputs 73 within the instrument handle 72. From the handle 72, the tendons are directed down one or more pull lumens along the elongated shaft 71 and anchored at the distal portion of the elongated shaft 71, or in the wrist at the distal portion of the elongated shaft. During a surgical procedure, such as a laparoscopic, endoscopic or hybrid procedure, these tendons may be coupled to a distally mounted end effector, such as a wrist, grasper, or scissor. Under such an arrangement, torque exerted on drive inputs 73 would transfer tension to the tendon, thereby causing the end effector to actuate in some way. In some embodiments, during a surgical procedure, the tendon may cause a joint to rotate about an axis, thereby causing the end effector to move in one direction or another. Alternatively, the tendon may be connected to one or more jaws of a grasper at distal end of the elongated shaft 71, where tension from the tendon cause the grasper to close.

In endoscopy, the tendons may be coupled to a bending or articulating section positioned along the elongated shaft 71 (e.g., at the distal end) via adhesive, control ring, or other mechanical fixation. When fixedly attached to the distal end of a bending section, torque exerted on drive inputs 73 would be transmitted down the tendons, causing the softer, bending section (sometimes referred to as the articulable section or region) to bend or articulate. Along the non-bending sections, it may be advantageous to spiral or helix the individual pull lumens that direct the individual tendons along (or inside) the walls of the endoscope shaft to balance the radial forces that result from tension in the pull wires. The angle of the spiraling and/or spacing there between may be altered or engineered for specific purposes, wherein tighter spiraling exhibits lesser shaft compression under load forces, while lower amounts of spiraling results in greater shaft compression under load forces, but also exhibits limits bending. On the other end of the spectrum, the pull lumens may be directed parallel to the longitudinal axis of the elongated shaft 71 to allow for controlled articulation in the desired bending or articulable sections.

In endoscopy, the elongated shaft 71 houses a number of components to assist with the robotic procedure. The shaft may comprise of a working channel for deploying surgical tools (or medical instruments), irrigation, and/or aspiration to the operative region at the distal end of the shaft 71. The shaft 71 may also accommodate wires and/or optical fibers to transfer signals to/from an optical assembly at the distal tip, which may include of an optical camera. The shaft 71 may also accommodate optical fibers to carry light from proximally-located light sources, such as light emitting diodes, to the distal end of the shaft.

At the distal end of the instrument 70, the distal tip may also comprise the opening of a working channel for delivering tools for diagnostic and/or therapy, irrigation, and aspiration to an operative site. The distal tip may also include a port for a camera, such as a fiberscope or a digital camera, to capture images of an internal anatomical space. Relatedly, the distal tip may also include ports for light sources for illuminating the anatomical space when using the camera.

In the example of FIG. 16, the drive shaft axes, and thus the drive input axes, are orthogonal to the axis of the elongated shaft. This arrangement, however, complicates roll capabilities for the elongated shaft 71. Rolling the elongated shaft 71 along its axis while keeping the drive inputs 73 static results in undesirable tangling of the tendons as they extend off the drive inputs 73 and enter pull lumens within the elongated shaft 71. The resulting entanglement of such tendons may disrupt any control algorithms intended to predict movement of the flexible elongated shaft during an endoscopic procedure.

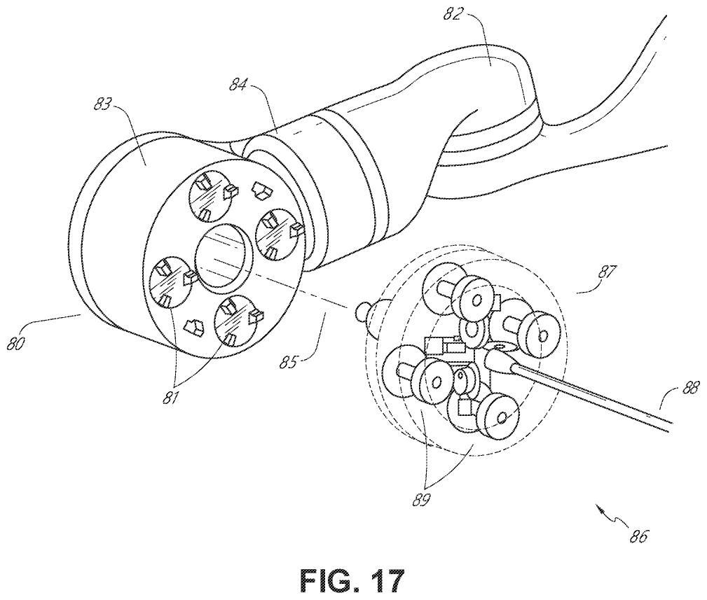

FIG. 17 illustrates an alternative design for an instrument driver and instrument where the axes of the drive units are parallel to the axis of the elongated shaft of the instrument. As shown, a circular instrument driver 80 comprises four drive units with their drive outputs 81 aligned in parallel at the end of a robotic arm 82. The drive units, and their respective drive outputs 81, are housed in a rotational assembly 83 of the instrument driver 80 that is driven by one of the drive units within the assembly 83. In response to torque provided by the rotational drive unit, the rotational assembly 83 rotates along a circular bearing that connects the rotational assembly 83 to the non-rotational portion 84 of the instrument driver. Power and controls signals may be communicated from the non-rotational portion 84 of the instrument driver 80 to the rotational assembly 83 through electrical contacts may be maintained through rotation by a brushed slip ring connection (not shown). In other embodiments, the rotational assembly 83 may be responsive to a separate drive unit that is integrated into the non-rotatable portion 84, and thus not in parallel to the other drive units. The rotational mechanism 83 allows the instrument driver 80 to rotate the drive units, and their respective drive outputs 81, as a single unit around an instrument driver axis 85.

Like earlier disclosed embodiments, an instrument 86 may comprise an elongated shaft portion 88 and an instrument base 87 (shown with a transparent external skin for discussion purposes) comprising a plurality of drive inputs 89 (such as receptacles, pulleys, and spools) that are configured to receive the drive outputs 81 in the instrument driver 80. Unlike prior disclosed embodiments, instrument shaft 88 extends from the center of instrument base 87 with an axis substantially parallel to the axes of the drive inputs 89, rather than orthogonal as in the design of FIG. 16.

When coupled to the rotational assembly 83 of the instrument driver 80, the medical instrument 86, comprising instrument base 87 and instrument shaft 88, rotates in combination with the rotational assembly 83 about the instrument driver axis 85. Since the instrument shaft 88 is positioned at the center of instrument base 87, the instrument shaft 88 is coaxial with instrument driver axis 85 when attached. Thus, rotation of the rotational assembly 83 causes the instrument shaft 88 to rotate about its own longitudinal axis. Moreover, as the instrument base 87 rotates with the instrument shaft 88, any tendons connected to the drive inputs 89 in the instrument base 87 are not tangled during rotation. Accordingly, the parallelism of the axes of the drive outputs 81, drive inputs 89, and instrument shaft 88 allows for the shaft rotation without tangling any control tendons.

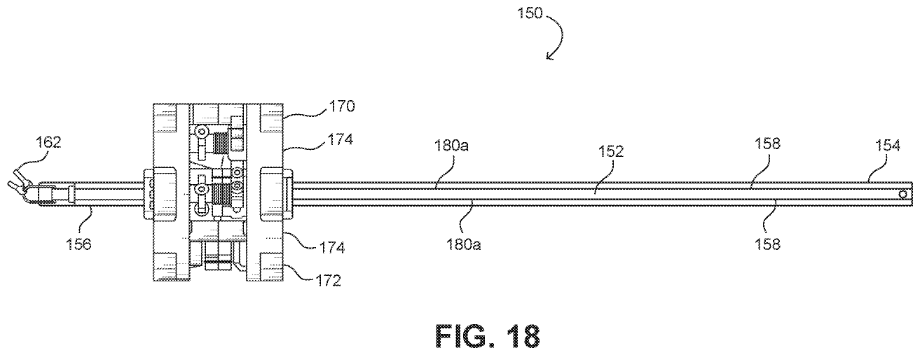

FIG. 18 illustrates an instrument having an instrument based insertion architecture in accordance with some embodiments. The instrument 150 can be coupled to any of the instrument drivers discussed above. The instrument 150 comprises an elongated shaft 152, an end effector 162 connected to the shaft 152, and a handle 170 coupled to the shaft 152. The elongated shaft 152 comprises a tubular member having a proximal portion 154 and a distal portion 156. The elongated shaft 152 comprises one or more channels or grooves 158 along its outer surface. The grooves 158 are configured to receive one or more wires or cables 180 therethrough. One or more cables 180 thus run along an outer surface of the elongated shaft 152. In other embodiments, cables 180 can also run through the elongated shaft 152. Manipulation of the one or more cables 180 (e.g., via an instrument driver) results in actuation of the end effector 162.

The instrument handle 170, which may also be referred to as an instrument base, may generally comprise an attachment interface 172 having one or more mechanical inputs 174, e.g., receptacles, pulleys or spools, that are designed to be reciprocally mated with one or more torque couplers on an attachment surface of an instrument driver.

In some embodiments, the instrument 150 comprises a series of pulleys or cables that enable the elongated shaft 152 to translate relative to the handle 170. In other words, the instrument 150 itself comprises an instrument-based insertion architecture that accommodates insertion of the instrument, thereby minimizing the reliance on a robot arm to provide insertion of the instrument 150. In other embodiments, a robotic arm can be largely responsible for instrument insertion.

### E. Controller.

Any of the robotic systems described herein can include an input device or controller for manipulating an instrument attached to a robotic arm. In some embodiments, the controller can be coupled (e.g., communicatively, electronically, electrically, wirelessly and/or mechanically) with an instrument such that manipulation of the controller causes a corresponding manipulation of the instrument e.g., via master slave control.

FIG. 19 is a perspective view of an embodiment of a controller 182. In the present embodiment, the controller 182 comprises a hybrid controller that can have both impedance and admittance control. In other embodiments, the controller 182 can utilize just impedance or passive control. In other embodiments, the controller 182 can utilize just admittance control. By being a hybrid controller, the controller 182 advantageously can have a lower perceived inertia while in use.

In the illustrated embodiment, the controller 182 is configured to allow manipulation of two medical instruments, and includes two handles 184. Each of the handles 184 is connected to a gimbal 186. Each gimbal 186 is connected to a positioning platform 188.

As shown in FIG. 19, each positioning platform 188 includes a SCARA arm (selective compliance assembly robot arm) 198 coupled to a column 194 by a prismatic joint 196. The prismatic joints 196 are configured to translate along the column 194 (e.g., along rails 197) to allow each of the handles 184 to be translated in the z-direction, providing a first degree of freedom. The SCARA arm 198 is configured to allow motion of the handle 184 in an x-y plane, providing two additional degrees of freedom.

In some embodiments, one or more load cells are positioned in the controller. For example, in some embodiments, a load cell (not shown) is positioned in the body of each of the gimbals 186. By providing a load cell, portions of the controller 182 are capable of operating under admittance control, thereby advantageously reducing the perceived inertia of the controller while in use. In some embodiments, the positioning platform 188 is configured for admittance control, while the gimbal 186 is configured for impedance control. In other embodiments, the gimbal 186 is configured for admittance control, while the positioning platform 188 is configured for impedance control. Accordingly, for some embodiments, the translational or positional degrees of freedom of the positioning platform 188 can rely on admittance control, while the rotational degrees of freedom of the gimbal 186 rely on impedance control.

### F. Navigation and Control.

Traditional endoscopy may involve the use of fluoroscopy (e.g., as may be delivered through a C-arm) and other forms of radiation-based imaging modalities to provide endoluminal guidance to an operator physician. In contrast, the robotic systems contemplated by this disclosure can provide for non-radiation-based navigational and localization means to reduce physician exposure to radiation and reduce the amount of equipment within the operating room. As used herein, the term “localization” may refer to determining and/or monitoring the position of objects in a reference coordinate system. Technologies such as pre-operative mapping, computer vision, real-time EM tracking, and robot command data may be used individually or in combination to achieve a radiation-free operating environment. In other cases, where radiation-based imaging modalities are still used, the pre-operative mapping, computer vision, real-time EM tracking, and robot command data may be used individually or in combination to improve upon the information obtained solely through radiation-based imaging modalities.

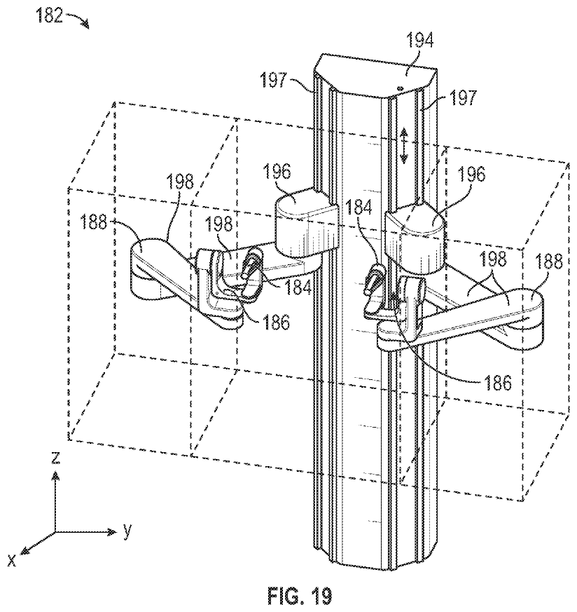

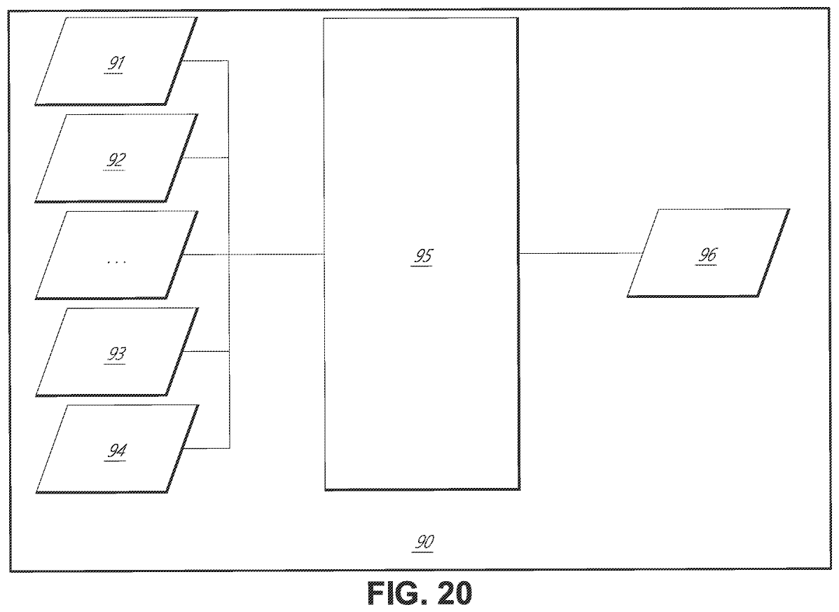

FIG. 20 is a block diagram illustrating a localization system 90 that estimates a location of one or more elements of the robotic system, such as the location of the instrument, in accordance to an example embodiment. The localization system 90 may be a set of one or more computer devices configured to execute one or more instructions. The computer devices may be embodied by a processor (or processors) and computer-readable memory in one or more components discussed above. By way of example and not limitation, the computer devices may be in the tower 30 shown in FIG. 1, the cart shown in FIGS. 1-4, the beds shown in FIGS. 5-14, etc.

As shown in FIG. 20, the localization system 90 may include a localization module 95 that processes input data 91-94 to generate location data 96 for the distal tip of a medical instrument. The location data 96 may be data or logic that represents a location and/or orientation of the distal end of the instrument relative to a frame of reference. The frame of reference can be a frame of reference relative to the anatomy of the patient or to a known object, such as an EM field generator (see discussion below for the EM field generator).

The various input data 91-94 are now described in greater detail. Pre-operative mapping may be accomplished through the use of the collection of low dose CT scans. Pre-operative CT scans are reconstructed into three-dimensional images, which are visualized, e.g. as “slices” of a cutaway view of the patient's internal anatomy. When analyzed in the aggregate, image-based models for anatomical cavities, spaces and structures of the patient's anatomy, such as a patient lung network, may be generated. Techniques such as center-line geometry may be determined and approximated from the CT images to develop a three-dimensional volume of the patient's anatomy, referred to as model data 91 (also referred to as “preoperative model data” when generated using only preoperative CT scans). The use of center-line geometry is discussed in U.S. patent application Ser. No. 14/523,760, the contents of which are herein incorporated in its entirety. Network topological models may also be derived from the CT-images, and are particularly appropriate for bronchoscopy.

In some embodiments, the instrument may be equipped with a camera to provide vision data 92. The localization module 95 may process the vision data to enable one or more vision-based location tracking. For example, the preoperative model data may be used in conjunction with the vision data 92 to enable computer vision-based tracking of the medical instrument (e.g., an endoscope or an instrument advance through a working channel of the endoscope). For example, using the preoperative model data 91, the robotic system may generate a library of expected endoscopic images from the model based on the expected path of travel of the endoscope, each image linked to a location within the model. Intra-operatively, this library may be referenced by the robotic system in order to compare real-time images captured at the camera (e.g., a camera at a distal end of the endoscope) to those in the image library to assist localization.

Other computer vision-based tracking techniques use feature tracking to determine motion of the camera, and thus the endoscope. Some features of the localization module 95 may identify circular geometries in the preoperative model data 91 that correspond to anatomical lumens and track the change of those geometries to determine which anatomical lumen was selected, as well as the relative rotational and/or translational motion of the camera. Use of a topological map may further enhance vision-based algorithms or techniques.

Optical flow, another computer vision-based technique, may analyze the displacement and translation of image pixels in a video sequence in the vision data 92 to infer camera movement. Examples of optical flow techniques may include motion detection, object segmentation calculations, luminance, motion compensated encoding, stereo disparity measurement, etc. Through the comparison of multiple frames over multiple iterations, movement and location of the camera (and thus the endoscope) may be determined.

The localization module 95 may use real-time EM tracking to generate a real-time location of the endoscope in a global coordinate system that may be registered to the patient's anatomy, represented by the preoperative model. In EM tracking, an EM sensor (or tracker) comprising of one or more sensor coils embedded in one or more locations and orientations in a medical instrument (e.g., an endoscopic tool) measures the variation in the EM field created by one or more static EM field generators positioned at a known location. The location information detected by the EM sensors is stored as EM data 93. The EM field generator (or transmitter), may be placed close to the patient to create a low intensity magnetic field that the embedded sensor may detect. The magnetic field induces small currents in the sensor coils of the EM sensor, which may be analyzed to determine the distance and angle between the EM sensor and the EM field generator. These distances and orientations may be intra-operatively “registered” to the patient anatomy (e.g., the preoperative model) in order to determine the geometric transformation that aligns a single location in the coordinate system with a position in the pre-operative model of the patient's anatomy. Once registered, an embedded EM tracker in one or more positions of the medical instrument (e.g., the distal tip of an endoscope) may provide real-time indications of the progression of the medical instrument through the patient's anatomy.

Robotic command and kinematics data 94 may also be used by the localization module 95 to provide localization data 96 for the robotic system. Device pitch and yaw resulting from articulation commands may be determined during pre-operative calibration. Intra-operatively, these calibration measurements may be used in combination with known insertion depth information to estimate the position of the instrument. Alternatively, these calculations may be analyzed in combination with EM, vision, and/or topological modeling to estimate the position of the medical instrument within the network.

As FIG. 20 shows, a number of other input data can be used by the localization module 95. For example, although not shown in FIG. 20, an instrument utilizing shape-sensing fiber can provide shape data that the localization module 95 can use to determine the location and shape of the instrument.

The localization module 95 may use the input data 91-94 in combination(s). In some cases, such a combination may use a probabilistic approach where the localization module 95 assigns a confidence weight to the location determined from each of the input data 91-94. Thus, where the EM data may not be reliable (as may be the case where there is EM interference) the confidence of the location determined by the EM data 93 can be decrease and the localization module 95 may rely more heavily on the vision data 92 and/or the robotic command and kinematics data 94.

As discussed above, the robotic systems discussed herein may be designed to incorporate a combination of one or more of the technologies above. The robotic system's computer-based control system, based in the tower, bed and/or cart, may store computer program instructions, for example, within a non-transitory computer-readable storage medium such as a persistent magnetic storage drive, solid state drive, or the like, that, upon execution, cause the system to receive and analyze sensor data and user commands, generate control signals throughout the system, and display the navigational and localization data, such as the position of the instrument within the global coordinate system, anatomical map, etc.

### 2. Cannula Reducers

Some embodiments disclosed herein provide a cannula reducer that may be utilized to support and align surgical instruments within a cannula. The cannula reducer can be coupled to a surgical instrument as an assembly and inserted into the cannula. Further, the cannula reducer can also or alternatively be coupled distal to a sealing layer in the cannula. Among the various advantages, such features can permit a clinician to leave a cannula in situ while switching between instruments of different size, thereby reducing trauma to the patient, decreasing surgery time, and reducing certain risks and other challenges associated with the additional work required using prior systems and methods. Such features may also or alternatively mitigate a need for secondary sealing mechanisms in the reducer, thereby lowering cost or complexity associated with the reducer device.

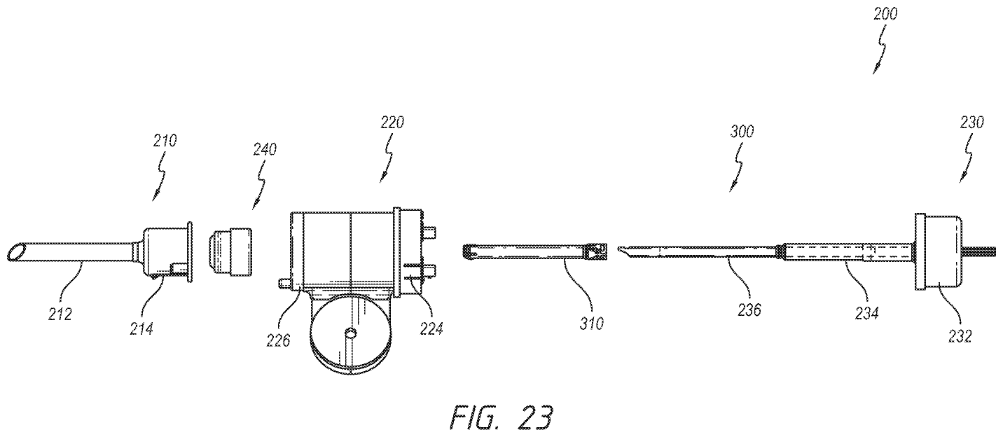

For example, FIGS. 21-23 depict an exemplary robotic system 200 for use with a cannula reducer 310 (shown in FIG. 23). According to some embodiments, the cannula reducer 310 may be utilized to support and align surgical instruments 230 within a cannula 210. In accordance with some embodiments described herein, the cannula reducer 310 may be configured to adapt an inner diameter of the cannula shaft 212 to support and coaxially align surgical instruments 230 configured or sized to be used with cannulas having a smaller inner diameter. The cannula reducer 310 can be used to adapt cannulas 210 and/or surgical instruments 230 configured for use with the systems 200 described herein.

In some embodiments, the cannula reducer 310 can attach to the surgical instrument 230, allowing the cannula reducer 310 and the surgical instrument 230 to be inserted into the cannula 210 as an assembled unit. Further, in some embodiments, the cannula reducer 310 can attach distal to all or a portion of a seal unit 240, allowing the seal unit 240 to directly seal against the surgical instrument 230.

FIG. 23 illustrates an exploded view of the robotic system 200. In the depicted example, the robotic system 200 includes a manipulator 220 configured to manipulate cannula 210 and a surgical instrument 230 inserted through the cannula 210. The manipulator 220 may be coupled to a distal end of a robotic arm and may be configured in accordance with any of the manipulators or instrument drivers described herein (e.g., 28, 62, 75, 80). The surgical instrument 230 may include an elongate instrument shaft 236 and may be configured in accordance with any of the surgical instruments described herein (e.g., 34, 70, 88, 150).

The manipulator 220 may be configured to dock to medical devices, such as the cannula 210 and/or the surgical instrument 230, such that the manipulator 220 may control movement of these devices. In the depicted example, the manipulator 220 includes an instrument dock 224, configured to attach to a base 232 of the surgical instrument 230, and a cannula dock 226, configured to attach to the funnel portion 214 of the cannula 210. In the illustrated embodiment, the instrument dock 224 is positioned on a first side of the manipulator 220 and the cannula dock 226 is positioned on a second side of the manipulator 220. The manipulator 220 further includes a passage 222 to accommodate the shaft 236 of the surgical instrument 230 and permit the shaft 236 of the surgical instrument 230 to extend through the manipulator 220. Each of the docks 224, 226 can facilitate docking and undocking of the respective device while securing the device to a body of the manipulator 220 when the device is in a docked configuration. In use, the cannula 210 may be inserted into a patient at a desired entry opening for the procedure. The manipulator 220 may then be moved to the position of the cannula 210, and the cannula dock 226 may be attached to the cannula 210 via a latching mechanism within the cannula dock 226. The instrument 230 may then be inserted through the cannula 210 and attached to the instrument dock 224. When in a docked configuration, the instrument 230 and cannula 210 may be coaxially aligned along an insertion axis such that the shaft 236 can extend through the passage 222 and the cannula 210. In some embodiments, the manipulator 220 can include one or more actuators to actuate or manipulate the instrument shaft 236 and/or an end effector 237 of the surgical instrument 230. For example, manipulator 220 can operate an actuation mechanism 244 of the instrument base to control the instrument shaft 236 and/or end effector 237 in one or more degrees of freedom.

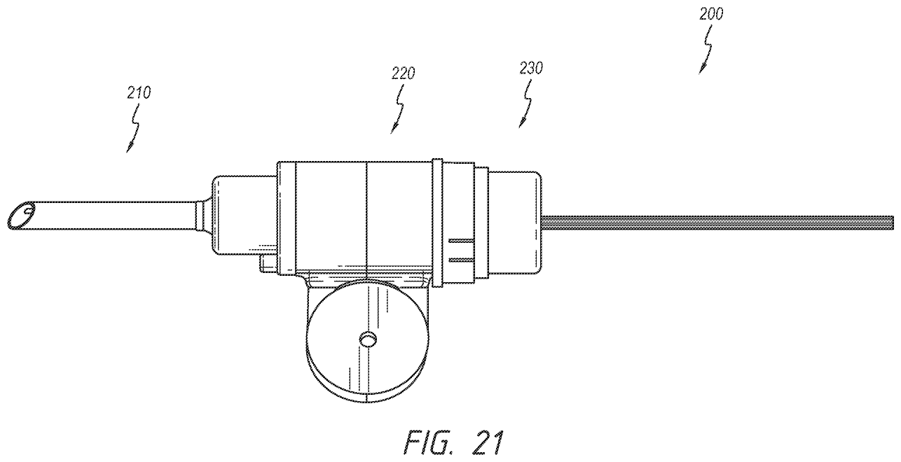

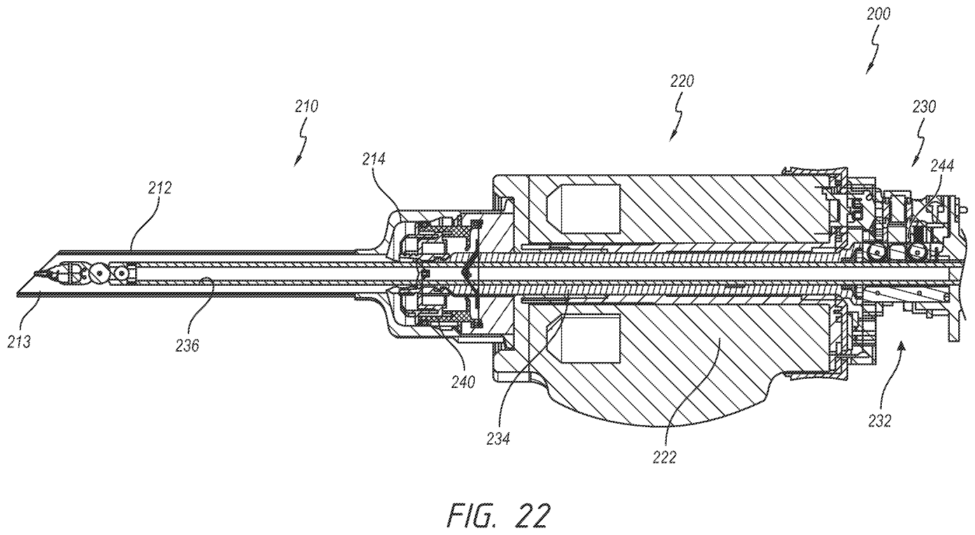

While FIG. 22 depicts a single manipulator docked to both the instrument 230 and the cannula 210, it is possible for multiple robotic manipulators or arms to be employed such that movement of the cannula 210 and the instrument 230 are controlled by separate robotic manipulator or arms. Further, the manipulator 220 may be configured to attach to the instrument 230 and the cannula 210 directly, or the manipulator 220 may be configured to attach to the instrument 230 and the cannula 210 through an intervening sterile barrier.

In the depicted example, the cannula 210 may displace or dissect soft tissue to allow the cannula 210 to be inserted into the patient cavity to provide access to a surgical site. Optionally, an obturator may be inserted through the cannula 210 during an initial insertion to provide a dilating or blunt tip that facilitates insertion of the cannula 210 through an incision or other opening on the patient. According to some embodiments, the cannula 210 is configured to provide access to a surgical site in the patient's abdomen for laparoscopic procedures. Additionally or alternatively, the cannula 210 can be configured to provide access to a site for urological, endoscopic, percutaneous, orthopedic, and/or or other medical or minimally invasive procedures in which a surgical instrument is introduced to the site through the cannula 210. In some applications, the cannula 210 provides access to a surgical site for robotic procedures performed by robotic systems described herein. Additionally or alternatively, the cannula 210 may be configured for use in manual procedures.

In the depicted example, a proximal portion of the cannula 210 is configured as a funnel portion 214, which has a generally larger diameter than the cannula shaft 212. The funnel portion 214 may transition to the smaller diameter cannula shaft 212 to facilitate insertion of tools from an opening at the proximal end of the cannula 210. Optionally, the funnel portion 214 of the cannula 210 can be utilized by clinicians or other users as a handle to advance the cannula 210 or to otherwise apply force to the cannula 210. In some embodiments, the funnel portion 214 may form an attachment point that can be engaged by a robotic manipulator to manipulate movement of the cannula 210. In some embodiments, the funnel portion 214 includes a seal unit 240, which can be fixed to the funnel portion or configured as a removable seal cartridge. The seal unit 240 can aid in sealing fluids, such as insufflation gas or liquids, within a patient cavity. A fluid port (such as a stop cock or luer fitting) may be positioned at the funnel portion 214 of the cannula 210 to provide connection point through which fluids may flow through the cannula 260 and into or out of the patient.

As illustrated, cannula shaft 212 is configured as a tubular portion that extends distally from the funnel portion 214. A lumen 213 of the cannula shaft 212 provides a passage through which a shaft of an instrument may extend to access a surgical site. The cannula lumen 213 can provide a working corridor or working channel through which tools can be inserted, manipulated, and/or removed along a longitudinal axis of the cannula. Optionally, the movement of the cannula 210 and the tools can be manipulated or controlled by robotic systems described herein. In the illustrated embodiment, the cannula shaft 212 is configured as a straight tube that terminates at a beveled distal end, but the cannula 210 and cannula shaft 212 may have a variety of configurations. For example, the cannula shaft 212 may be curved, bent, or angled, or the distal end of the cannula shaft 212 may be flat or have other geometries.

As described herein, the cannula 210 can be used with the surgical instrument 230 and configured to provide access the patient's cavity, even though the cannula shaft 212 has an inner diameter that is larger than the outer diameter of the instrument shaft 236. In some embodiments, the lumen 213 of the cannula shaft 212 can support and coaxially align the shaft 236 of the surgical instrument 230 within the cannula shaft 212. In some applications, the cannula 210 is sized or configured to support and/or align a surgical instrument 230 of a certain size or diameter. For example, the cannula 210 may be sized or configured to support and/or align a surgical instrument 230 having a shaft 236 with a nominal 12 mm diameter.

In certain laparoscopic procedures, a clinician may utilize a cannula configured to be used with 12 mm diameter surgical instruments with surgical instruments having 8 mm diameter shafts, in order to provide flexibility in instrument access and minimize the number of incisions needed for unique cannulas. However, in certain applications, such as robotically assisted laparoscopic procedures, extra clearance between an 8 mm diameter instrument and the working corridor of a 12 mm diameter cannula can allow for misalignment between the instrument's centerline and the cannula's centerline, which may reduce the positional accuracy of the instrument's working end. In some applications, a robotic surgical system may be not be able to compensate for the reduction in positional accuracy.

Advantageously, the cannula reducer can be used to reduce the inner profile or inner diameter into which an instrument is be fitted when the inner diameter of the cannula is otherwise too large or intended to be used with instruments having a larger outer profile or outer diameter, thus allowing the instrument's shaft to be supported and aligned with the cannula intended for a larger surgical instrument. In some applications, certain conventional reducers may not be compatible with the docking arrangement of the cannula 210, the surgical instrument 230 and/or the manipulator 220 described herein. Further, certain conventional reducers may couple or attach to the cannula, requiring that the surgical instrument is first removed from the cannula in a separate step in order to introduce or remove the reducer. Further, certain conventional reducers may pass through a seal unit, reducing the effectiveness of the seal unit and requiring a secondary sealing mechanism or sealing surface to maintain insufflation.

Therefore, as noted above, in accordance with at least one embodiment disclosed herein is the realization that it may be desirable to provide a cannula reducer that can adapt the inner diameter of the cannula while being compatible with the docking arrangement of the cannula, the surgical instrument, and/or the manipulator. Further, in accordance with at least one embodiment disclosed herein is the realization that it may be desirable to provide a cannula reducer that can attach to the surgical instrument, allowing the cannula reducer and the surgical instrument to be handled and/or inserted into the cannula as an assembled unit. Further, in accordance with at least one embodiment disclosed herein is the realization that it may be desirable to provide a cannula reducer that can attach distal to a sealing layer, allowing the sealing layer to directly seal against the surgical instrument. The various embodiments disclosed herein allow a clinician to achieve these and other advantageous outcomes, as discussed herein.

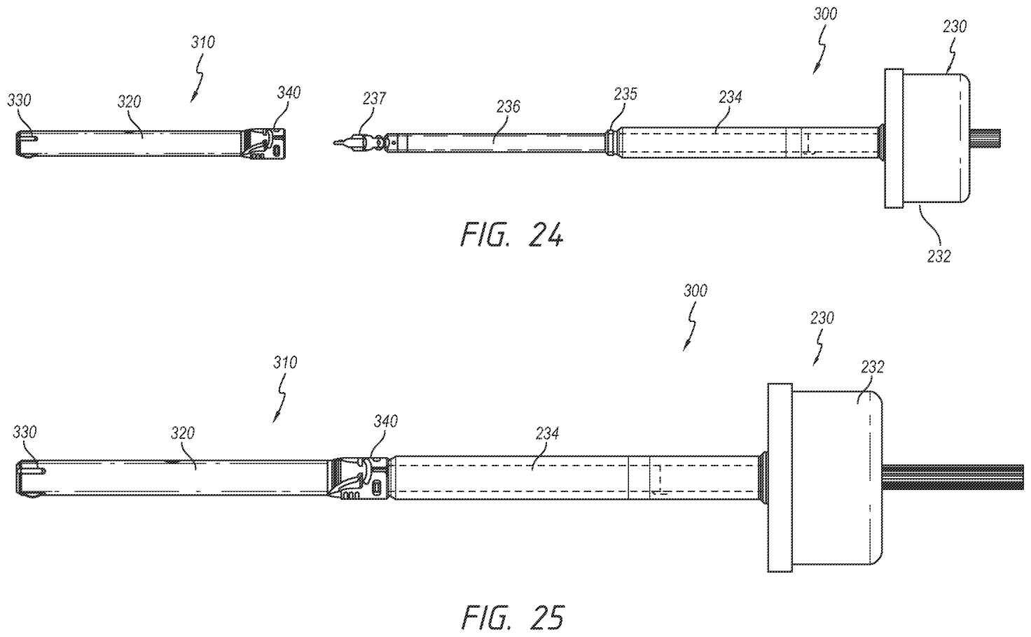

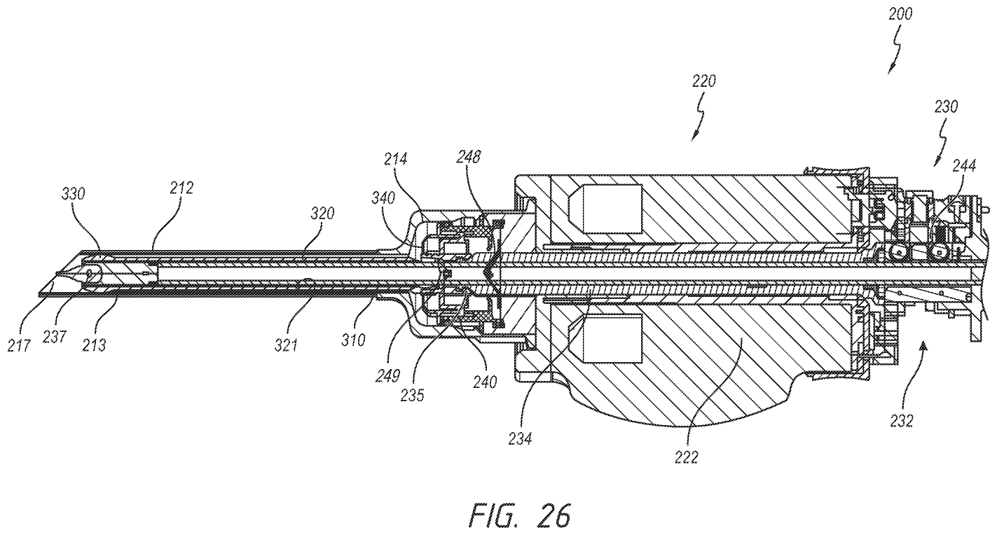

FIGS. 24 and 25 depict a cannula reducer 310 and a surgical instrument 230. FIG. 26 depicts the cannula reducer 310 and the surgical instrument 230 docked within a surgical system. In the depicted example, the cannula reducer 310 can be attached to the surgical instrument 230 to adapt the surgical instrument 230 to securely fit and/or align within a cannula 210 intended for a larger surgical instrument.

As illustrated, the cannula reducer 310 has a reducer sleeve or body 320 generally shaped to securely fit and/or align within a cannula 210. In some embodiments, the reducer body 320 has a generally tubular or cylindrical shape. Optionally, the reducer body 320 can be keyed, splined, bent, curved, etc. to fit within the cannula 210.

In the depicted example, the reducer body 320 has an outer diameter configured to securely fit and/or align within a cannula 210. In the depicted example, the outer diameter of the reducer body 320 can be sized or configured to be supported or aligned by the cannula lumen 213. For example, the outer diameter of the reducer body 320 can have a nominal 12 mm diameter to be supported and/or aligned within a cannula configured for use with 12 mm diameter surgical instruments. The outer diameter of the reducer body 320 can be any suitable size to correspond to the working corridor or lumen diameter of the desired cannula to allow the cannula reducer 310 to be fully supported and/or aligned within the cannula. In the depicted example, the reducer body 320 has an outer diameter that is greater than the outer diameter of the instrument shaft 236.

As illustrated, the reducer body 320 can have a hollow or tubular structure defining a reducer passage 321 to receive an instrument shaft 236 of the surgical instrument 230. In the depicted example, the reducer passage 321 is a hollow portion of the reducer body 320 defining a lumen extending through an interior of the reducer body 320, and can be generally shaped to receive, fit, and align the instrument shaft 236 of the surgical instrument 230. The reducer passage 321 can have a generally circular cross-sectional shape. Optionally, the reducer passage 321 can be keyed, bent, curved, etc. to mate with the instrument shaft 236. The instrument shaft 236 can be inserted into the reducer passage 321 through a proximal end 340 of the cannula reducer 310.

In the depicted example, the inner diameter of the reducer passage 321 is sized or configured to support and/or axially align the instrument shaft 236. For example, the inner diameter of the reducer passage 321 can be configured to receive instrument shafts 236 having a nominal 8 mm diameter. The inner diameter of the reducer passage 321 can be any suitable size to provide a working corridor that corresponds to the nominal diameter of a desired surgical instrument 230 that allows the instrument shaft 236 to be fully supported and/or aligned within the cannula reducer 310. Advantageously, by supporting and/or aligning the surgical instrument 230 within the cannula reducer 310 and supporting and/or aligning the cannula reducer 310 within the cannula 210, the surgical instrument 230 can be supported and/or aligned within the cannula 210, even in applications wherein the cannula 210 is intended to be used with a surgical instrument with a larger nominal diameter (e.g. an 8 mm diameter surgical instrument with a 12 mm diameter cannula).

As illustrated, the cannula reducer 310 and the surgical instrument 230 can be attached together as an assembled unit 300. Advantageously, the cannula reducer 310 and the surgical instrument 230 can be inserted, coupled, or docked with the manipulator 220 and/or inserted into the cannula 210 as an assembled unit 300. Further, because the cannula reducer 310 can be attached to the surgical instrument 230, the surgical instrument 230 and the cannula reducer 310 can be removed as an assembled unit 300, facilitating rapid exchanges of surgical instruments 230 from the cannula 210 and/or the manipulator 220. Upon removal of the assembled unit 300 from the cannula 210 and/or the manipulator 220, the cannula reducer 310 can be removed from the surgical instrument 230.

As illustrated, a proximal end 340 of the cannula reducer 310 can be coupled or attached to the surgical instrument 230. For example, the proximal end 340 of the cannula reducer 310 can attach to various features of the surgical instrument 230 positioned around the instrument shaft along the length of the instrument shaft 236. In the depicted example, the proximal end 340 of the cannula reducer 310 can attach to various features on an outer portion of the surgical instrument 230.

In some embodiments, the proximal end 340 is coupled or attached to a base portion 232 of the surgical instrument 230. A housing of the base portion 232 can house actuation or drive mechanisms used to actuate the instrument shaft 236 and/or the end effector 237 of the surgical instrument 230, and can extend over a portion of the instrument shaft 236. In the depicted example, the base portion 232 includes a proximal housing portion that can define a main body and handle of the instrument where actuation mechanisms are supported, and a distal housing portion that defines an instrument sleeve 234 extending from the main body over a portion of the instrument shaft 236. In the depicted example, the proximal end 340 of the cannula reducer 310 attaches to a distal end of the instrument sleeve 234. Optionally, the base portion 232 and/or the instrument sleeve 234 can allow the surgical instrument 230 to be aligned within a cannula and/or relative to the cannula reducer.

FIG. 26 illustrates a cross section view of surgical instrument 230, manipulator 220, cannula 210, and reducer 310 in a docked configuration. FIG. 26 also depicts actuation mechanism 244 of the instrument, which can be operated by manipulator 220 to actuate portions of the instrument 230. Actuation mechanism 244 can include an internal assembly of components such as one or more pulleys, spools, and/or gears, that can be operated by the manipulator 220 to actuate one or more degrees of freedom of the instrument shaft 236. In some embodiments, the actuation mechanism 244 can be operated to drive movement of the instrument shaft 236 relative to portions of the instrument base 232, such that the instrument shaft 236 moves with respect to the cannula 210, instrument sleeve 234, and the attached reducer 310 when the actuation mechanism 244 is operated. For example, operation of actuation mechanism 244 can be configured to drive axial movement of the instrument shaft 236 relative to the instrument base 232, such that the instrument shaft 236 is advanced or retracted through the instrument sleeve 234 and through the reducer 310. Accordingly, the reducer 310 attached to the surgical instrument 230 may be maintained in position within the cannula 210 and with respect to the instrument base 232 as the instrument shaft 236 is manipulated and moved through the cannula 210, the reducer 310, and the instrument base 232 to perform a medical procedure.

As illustrated in FIG. 26, the cannula reducer 310 and/or the surgical instrument 230 can be sized or otherwise configured such that when the cannula reducer 310 and the surgical instrument 230 are inserted into or docked with the cannula 210, the proximal end 340 of the cannula reducer 310 can be positioned distal to a sealing layer disposed within the seal unit 240. Here, the length of the instrument sleeve 234 and the spacing between the instrument dock 224 and cannula dock 226 of the manipulator 220 are such that, when the instrument 230 and cannula 210 are both docked to the manipulator 220, the distal end of the instrument sleeve 234 extends into the funnel portion 214 of the cannula 210 beyond a proximal sealing layer 248 positioned in the cannula 210. The proximal sealing layer 248 may define a sealing plane such that, when the proximal end of the reducer 310 is attached to the distal end of the instrument sleeve 234, the position of the proximal end of the reducer 310 and the attachment point between the reducer 310 and the instrument sleeve 234 is distal to the sealing plane. Optionally, the proximal end 340 of the cannula reducer 310 can be positioned distal to both a distal sealing layer 249 and a proximal sealing layer 248 within the seal unit 240 in the docked configuration. In some embodiments, the proximal end 340 of the cannula reducer 310 can be positioned between the distal sealing layer 249 and the proximal sealing layer 248 within the seal unit 240 in the docked configuration. In such embodiments, the proximal sealing layer 248 may provide a seal against the instrument 230 when the instrument 230 is present in the cannula 210 while the distal sealing layer 249 may provide a seal when no instrument is present in the cannula 210. Advantageously, by positioning the proximal end 340 of the cannula reducer 310 distal to sealing layers within the seal unit 240, the use of the cannula reducer 310 does not affect the engagement or seal of the sealing layers against the surgical instrument 230 and therefore allows the seal unit 240 to maintain insufflation.

Optionally, the cannula reducer 310 and/or the surgical instrument 230 can be sized or otherwise configured such that when the cannula reducer 310 and the surgical instrument 230 are inserted into or docked with the cannula 210, the proximal end 340 of the cannula reducer 310 can be positioned proximal to the seal unit 240 or a sealing layer disposed within the seal unit 240. For example, the reducer 310 may be configured to attach to other portions of the surgical instrument 230, such as the main body portion of the instrument base housing, or to a location along the instrument sleeve 234 that is proximal to the sealing layer. In another example, the distal end of the instrument sleeve 234 may terminate proximal to the sealing layer when the instrument is inserted into the cannula.

In the depicted example, the cannula reducer 310 and/or the surgical instrument 230 can be sized or otherwise configured such that when the cannula reducer 310 and the surgical instrument 230 are joined as the assembled unit 300, the distal end 330 of the cannula reducer can be positioned proximal to the end effector 237 of the surgical instrument 230. Further, the cannula reducer 310 and/or the surgical instrument 230 can be sized or otherwise configured such that when the cannula reducer 310 and the surgical instrument 230 are inserted into or docked with the cannula 210, the distal end 330 of the cannula reducer 310 can be positioned proximal to a distal end 217 of the cannula 210.

As used herein, the terms “proximal end” and/or “distal end” can refer to the end points of a body or component, as well as to the portions that are adjacent, near, or otherwise in proximity with the end points of the body or component.

For example, the distal end 330 and/or proximal end 340 can refer to the end points of the reducer body 320 as well as to the portions of the reducer body that are adjacent, near, or otherwise in proximity with the end points of the reducer body 320. Therefore, the term proximal end 340 can include portions of the reducer body 320 that are near or otherwise adjacent to the proximal end point of the reducer body 320 and the term distal end 330 can include portions of the reducer body 320 that are near or otherwise adjacent to the distal end point of the reducer body 320.

The cannula reducer can utilize various types of attachment mechanisms, such as mechanical latches, magnetic latches, or threaded connections to releasably engage the cannula reducer to the surgical instrument. FIGS. 27 and 28 depict an example of a latch mechanism that the cannula reducer 310 can utilize to releasably attach, engage or latch to the surgical instrument 230 to secure the cannula reducer 310 to the surgical instrument 230. As described herein, the cannula reducer 310 can latch to the base portion 232 and/or the instrument sleeve 234 of the surgical instrument 230. In the depicted example, the cannula reducer 310 can latch to a feature or receptacle of the surgical instrument 230.

As illustrated, the latching mechanism can include one or more latch members 342 that can engage with a feature or lip 235 extending from the surface of the instrument sleeve 234. In some embodiments, the lip 235 can extend or protrude from a distal portion or any other suitable portion of the surgical instrument 230. In the depicted example, the latch members 342 can include a groove, recess, or other complimentary feature that receives a portion of the lip 235. The engagement of the latch members 342 with the lip 235 can limit or prevent axial movement of the cannula reducer 310 relative to a portion of the surgical instrument 230. In some embodiments, the engagement of the latch members 342 with the lip 235 can allow the cannula reducer 310 to rotate relative to a portion of the surgical instrument 230.

In the depicted example, the latch members 342 can move to engage and disengage from the lip 235. For example, the latch members 342 may be moved radially inward to engage against the lip 235 and retain the cannula reducer 310 relative to the surgical instrument 230. The latch members 342 may biased inward to engage the lip 235. The latch members 342 can be moved radially outward to disengage the lip 235 and release the cannula reducer 310 from the surgical instrument 230.

As illustrated, the latch members 342 can move or pivot relative to the reducer body 320 to allow the latch members 342 to engage or disengage the lip 235. The latch members 342 can be coupled to the reducer body 320 with one or more hinge portions 346 that allow the latch members 342 to move relative to the reducer body 320. The hinge portions 346 may be biased to urge the latch members 342 to engage with the lip 235.

In the depicted example, the hinge portions 346 can be a living hinge or flexural portion that deflects or deforms to allow the latch members 342 to move. In some embodiments, the latch members 342 and the hinge portions 346 can be integrally or monolithically formed with the reducer body 320, reducing the complexity of the cannula reducer 310. Optionally, the hinge portions 346 can have a different thickness, construction, or material composition relative to the reducer body 320 to allow the latch members 342 to move in a desired manner. In some embodiments, the latch members 342 can be a separate portion that is attached to the reducer body 320.

Optionally, the latch members 342 can be actuated by depressing one or more release buttons 344. The release buttons 344 provide an actuation portion that can be coupled to the latch members 342 to allow the latch members 342 to move in response to actuation of the release buttons 344. For example, a clinician may depress the release buttons 344 to disengage the latch members 342 from the lip 235. The release buttons 344 can have grooves or other features to allow a clinician to actuate the release buttons 344.

In some embodiments, the release button 344 and the latch member 342 can rotate about a common pivot point, such as hinge portion 346. Therefore, as the release button 344 is depressed radially inward, the latch member 342 may be moved radially outward to disengage the latch members 342 from the lip 235. Similarly, as the latch members 342 move radially inward to engage the lip 235, the release buttons 344 can move radially outward. Similar to the latch members 342, the release buttons 344 can be integrally or monolithically formed with the reducer body 320, reducing the complexity of the cannula reducer 310.

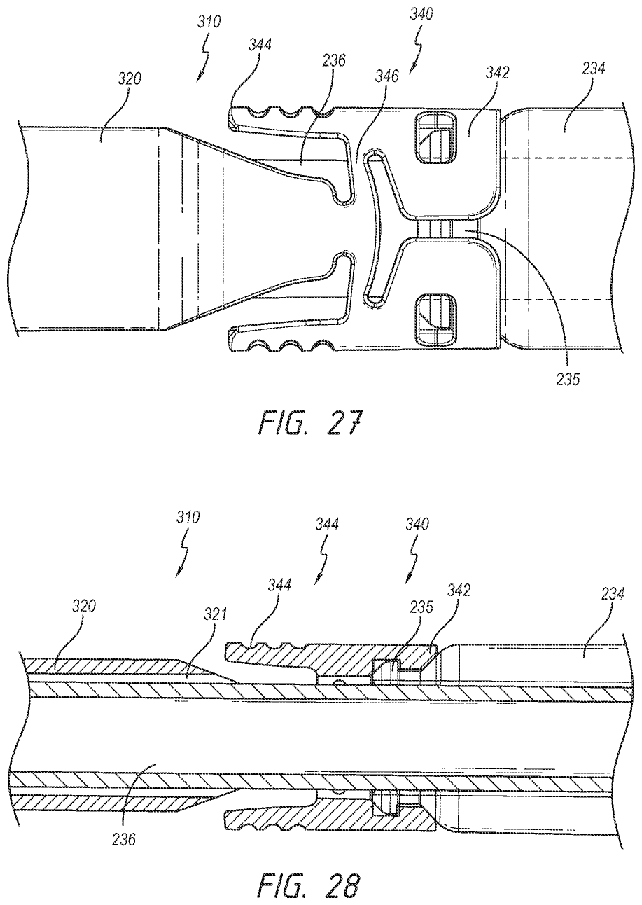

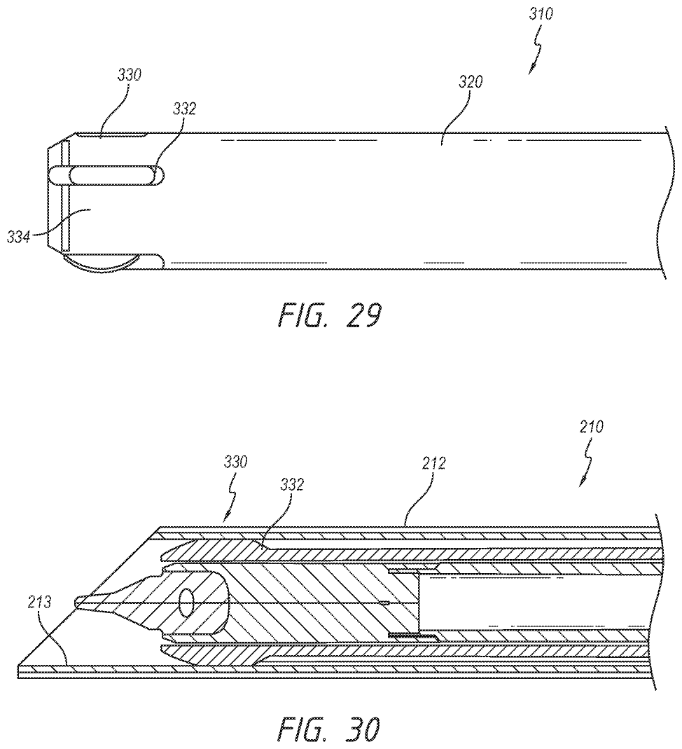

With reference to FIGS. 29 and 30, in some embodiments, the distal end 330 of the cannula reducer 310 can include one or more ribs 332 to create a flow path allowing insufflation gas to pass between the cannula reducer 310 and the lumen 213 of the cannula 210. As illustrated, the ribs 332 can radially extend from a surface at the distal end 330 of the reducer body 320. In some embodiments, the ribs 332 can be configured to extend to contact an inner surface of the cannula lumen 213. The ribs 332 can be circumferentially spaced apart. Optionally, the ribs 332 can be equidistantly spaced apart. The cannula reducer 310 can include any suitable number of ribs 332, including, but not limited to two, four, six, or eight ribs. The ribs 332 can have any suitable shape or cross-sectional profile. In some embodiments, the ribs 332 can be defined as grooves or cut outs in the reducer body 320.

As illustrated, the spacing between the ribs 332 defines radial gaps 334 that permit fluid flow between the outer surface of the reducer body 320 and the inner surface of the cannula lumen 213. In addition to permitting fluid flow, the ribs 332 can provide structural support to the cannula 210.

With reference to FIG. 31, a cannula reducer 410 can include one or more latch members 442 that can engage through a window 235′ defined in the surface of the instrument sleeve 234 to form an assembled unit 400. In some embodiments, the window 235′ can be defined in a distal portion or any other suitable portion of the surgical instrument 230. In the depicted example, the latch members 442 can include a radially protruding portion that extends through and/or engages with the edges of the window 235′. The engagement of the latch members 442 with the window 235′ can limit or prevent axial and/or rotational movement of the cannula reducer 410 relative to the surgical instrument 230.

In the depicted example, the latch members 442 can move to engage and disengage from the window 235′. For example, the latch members 442 may be moved radially outward to extend through the window 235′ and retain the cannula reducer 410 relative to the surgical instrument 230. The latch members 442 may biased outward to engage the window 235′. The latch members 442 can be moved radially inward to disengage the window 235′ and release the cannula reducer 410 from the surgical instrument 230.

As illustrated, the latch members 442 can move or pivot relative to the reducer body 420 to allow the latch members 442 to engage or disengage the window 235′. The latching members 442 can be coupled to the reducer body 420 with one or more hinge portions 446 that allow the latch members 442 to move relative to the reducer body 420. The hinge portions 446 may be biased to urge the latching members 442 to engage with the window 235′.

As described herein, the hinge portions 446 can be a living hinge or flexural portion that deflects or deforms to allow the latch members 442 to move. Optionally, the latch members 442 themselves provide release buttons that can be actuated by depressing directly on the latch members 442. For example, a clinician may depress the latch members 442 to disengage the latch members 442 from the windows 235′. The latch members 442 can have grooves or other features to allow a clinician to actuate the latch members 442.

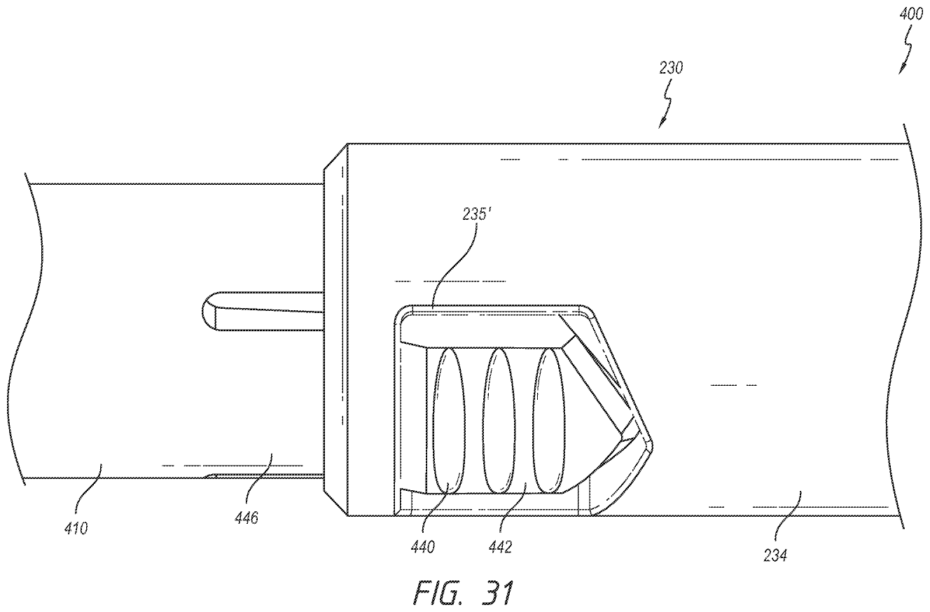

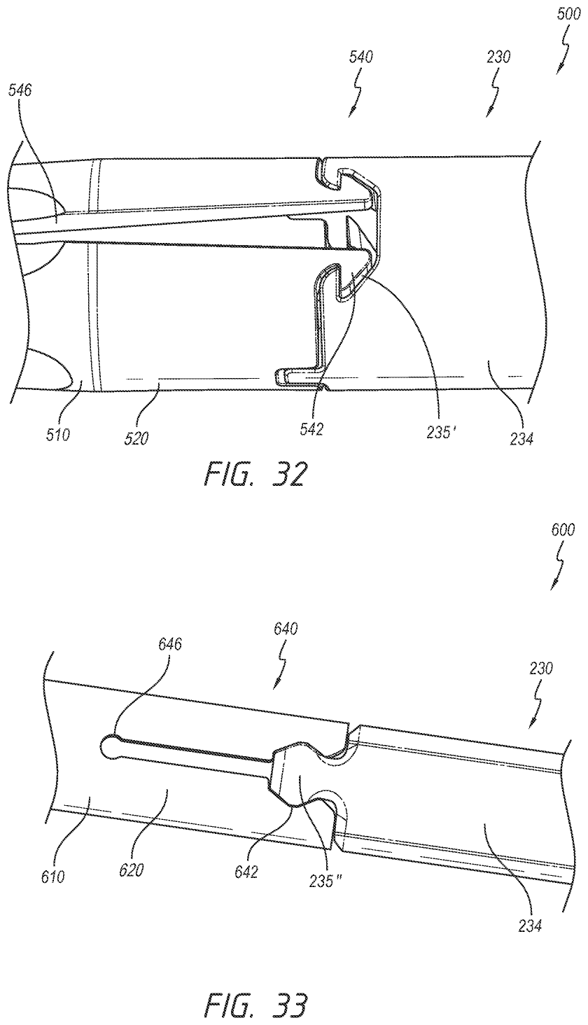

With reference to FIG. 32, a cannula reducer 510 can include a latching mechanism with an opposing latching finger arrangement to releasably latch to a receptacle of the surgical instrument 230 to form an assembled unit 500. As illustrated, the cannula reducer 510 can include one or more latch members 542 configured as latching fingers that can move outward along the circumference of the cannula reducer 510 and relative to each other to engage the edges of a window 235′ defined in the surface of the instrument sleeve 234. In some embodiments, the window 235′ can be defined in a distal portion or any other suitable portion of the surgical instrument 230. In the depicted example, the latching fingers can include hooked portions that extend through and/or engage with the edges of the window 235′. The engagement of the latching fingers with the window 235′ can limit or prevent axial and/or rotational movement of the cannula reducer 510 relative to a portion of the surgical instrument 230.

In the depicted example, the latching fingers can move to engage and disengage from the window 235′. For example, the latching fingers may be moved outward relative to each other to engage the edges of the window 235′ and retain the cannula reducer 510 relative to the surgical instrument 230. The latching fingers may be biased outward relative to each other to engage the edges of the window 235′. The latching fingers can provide release buttons that are moved inward toward each other to disengage the window 235′ and release the cannula reducer 510 from the surgical instrument 230.

As illustrated, the latching fingers can move or pivot relative to each other and the reducer body 520 to allow the latching fingers to engage or disengage the window 235′. The latching fingers can be coupled to the reducer body 520 with one or more hinge portions 546 that allow the latching fingers to move relative to each other and the reducer body 520. The hinge portions 546 may be biased to urge the latching fingers outward relative to each other to engage with the window 235′.

As described herein, the hinge portions 546 can be a living hinge or flexural portion that deflects or deforms to allow the latching fingers to move. Optionally, the latching fingers can provide release buttons that are actuated by squeezing the proximal end 530 of the reducer body 520. For example, a clinician may squeeze the proximal end 530 of the reducer body 520 to disengage the latching fingers from the windows 235′.

With reference to FIG. 33, a cannula reducer 610 can include a latching mechanism with an opposing latching finger arrangement to releasably latch to a protruding feature of the surgical instrument 230 to form an assembled unit 600. As illustrated, the cannula reducer 610 can include one or more latch members 642 configured as latching fingers that can engage the edges of a protrusion 235″ extending from a distal end of the instrument sleeve 234. Here, the latch members 642 may disengage from the instrument 230 without actuating release buttons by pulling the reducer body 620 apart from the instrument 230 with sufficient force. In the depicted example, the latching fingers can include hooked portions with a mating profile that engages with the edges of the protrusion 235″. The engagement of the latching fingers with the protrusion 235″ can limit or prevent axial and/or rotational movement of the cannula reducer 610 relative to a portion (e.g., sleeve 234) of the surgical instrument 230.

In the depicted example, the latching fingers can move to engage and disengage from the protrusion 235″. For example, the latching fingers may be moved inward relative to each other to engage the edges of the protrusion 235″ and retain the cannula reducer 610 relative to the surgical instrument 230. The latching fingers may be biased inward to engage the edges of the protrusion 235″. The latching fingers can be moved outward away from each other to disengage the protrusion 235″ and release the cannula reducer 610 from the surgical instrument 230.

As illustrated, the latching fingers can move or pivot relative to each other and the reducer body 620 to allow the latching fingers to engage or disengage the protrusion 235″. The latching fingers can be coupled to the reducer body 620 with one or more hinge portions 646 that allow the latching fingers to move relative to each other and the reducer body 620. The hinge portions 646 may be biased to urge the latching fingers inward relative to each other to engage with the protrusion 235″.

As described herein, the hinge portions 646 can be a living hinge or flexural portion that deflects or deforms to allow the latching fingers to move. Optionally, the latching fingers can be deflected by the insertion or removal of the protrusion 235″ from the latching fingers. Accordingly, the latching fingers can retain the surgical instrument 230 with a predetermined force and allow the cannula reducer 610 to be released from the surgical instrument 230 if the predetermined force is exceeded.

In some embodiments, a cannula reducer can be laterally attached to a surgical instrument prior to insertion within a cannula. Laterally attached cannula reducers may allow a clinician to securely attach the cannula reducer to the surgical instrument without introducing a risk of injury to the clinician or damage to the surgical instrument by avoiding contact with the end effector or other portions of the surgical instrument.

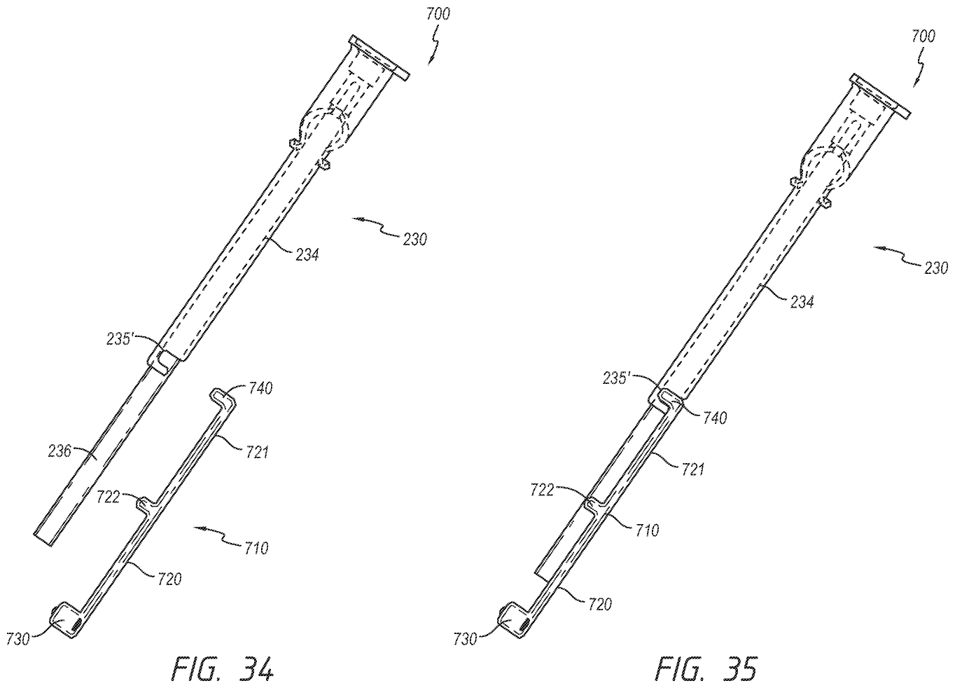

For example, with reference to FIGS. 34 and 35, a cannula reducer 710 can be laterally attached to the surgical instrument 230 to adapt the surgical instrument 230 to securely fit and/or align within a cannula intended for a larger surgical instrument. As illustrated, the cannula reducer 710 has a reducer body 720 with a proximal end 740, a medial portion 722, and a distal end 730 generally shaped to securely fit and/or align within a cannula. In some embodiments, the proximal end 740, the medial portion 722, and the distal end 730 are configured to contact portions of the cannula lumen.

As illustrated, the proximal end 740 and the medial portion 722 can each and cooperatively define a lateral channel 721 to receive an instrument shaft 236 of the surgical instrument 230. In the depicted example, the lateral channel 721 can be generally shaped to receive, fit, and align the instrument shaft 236 of the surgical instrument 230. The lateral channel 721 can have a generally “C” shaped cross-sectional shape with a channel opening extending between the proximal end 740 and the distal end 730 to receive the instrument shaft 236. The instrument shaft 236 can be laterally inserted into the lateral channel 721 through the channel opening extending between the proximal end 740 and the distal end 730. In some embodiments, the distal end 730 may form a closed loop, and the instrument shaft 236 of the surgical instrument 230 can be retracted during insertion to avoid contact with the distal end 730 during lateral attachment.

In the depicted example, the inner dimensions of the lateral channel 721 is sized or configured to support and/or axially align the instrument shaft 236. As described herein, the cannula reducer 710 and the surgical instrument 230 can be attached together as an assembled unit 700.

As illustrated, the proximal end 740 of the cannula reducer 710 can be coupled or attached to the surgical instrument 230. In the depicted example, the cannula reducer 710 can releasably attach, engage or latch to the surgical instrument 230 to secure the cannula reducer 710 to the surgical instrument 230.

As illustrated, the proximal end 740 can engage with a feature or window 235′ defined in the surface of the instrument sleeve 234. In some embodiments, the window 235′ is a keyed slot defined in a distal portion of instrument sleeve 234 or any other suitable portion of the surgical instrument 230. In the depicted example, the proximal end 740 defines a circumferentially extending portion that can extend through and/or engage with the edges of the window 235′. The engagement of the proximal end 740 with the window 235′ can limit or prevent axial movement of the cannula reducer 710 relative to the surgical instrument 230.

In the depicted example, the proximal end 740 can move to engage and disengage from the window 235′. For example, the proximal end 740 can snap, deform, or have an interference fit with the window 235′ to retain the cannula reducer 710 relative to the surgical instrument 230. The proximal end 740 may be deformed to disengage the window 235′ and release the cannula reducer 710 from the surgical instrument 230.

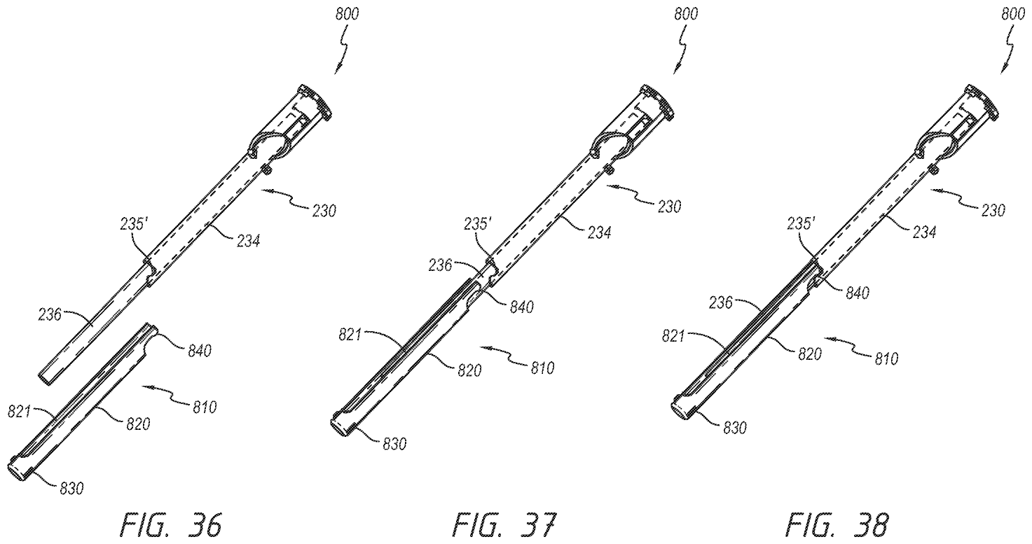

With reference to FIGS. 36-38, a cannula reducer 810 can be laterally attached to the surgical instrument 230 to adapt the surgical instrument 230 to securely fit and/or align within a cannula intended for a larger surgical instrument.

As illustrated, the cannula reducer 810 has a reducer sleeve or body 820 with a proximal end 840 and a distal end 830 generally shaped to securely fit and/or align within a cannula. In some embodiments, the reducer body 820 has a generally tubular or cylindrical shape. Optionally, the reducer body 820 can be keyed, splined, bent, curved, etc. to fit within the cannula.

In the depicted example, the reducer body 820 has an outer diameter configured to securely fit and/or align within a cannula. In the depicted example, the outer diameter of the reducer body 820 can be sized or configured to be supported or aligned by the cannula lumen.

As illustrated, the reducer body 820 can define a lateral channel 821 to receive an instrument shaft 236 of the surgical instrument 230. In the depicted example, the lateral channel 821 can be generally shaped to receive, fit, and align the instrument shaft 236 of the surgical instrument 230. The lateral channel 821 can have a generally “C” shaped cross-sectional shape with a channel opening extending between the proximal end 840 and the distal end 830 to receive the instrument shaft 236. The instrument shaft 236 can be laterally inserted into the lateral channel 821 through the channel opening extending between the proximal end 840 and the distal end 830. As illustrated, the proximal end 840 can engage with a feature or window 235′ defined in the surface of the instrument sleeve 234. Here, the lateral attachment sequence involves axially moving the reducer 810 to engage the surgical instrument after laterally loading the instrument shaft into the passage of the reducer 810.

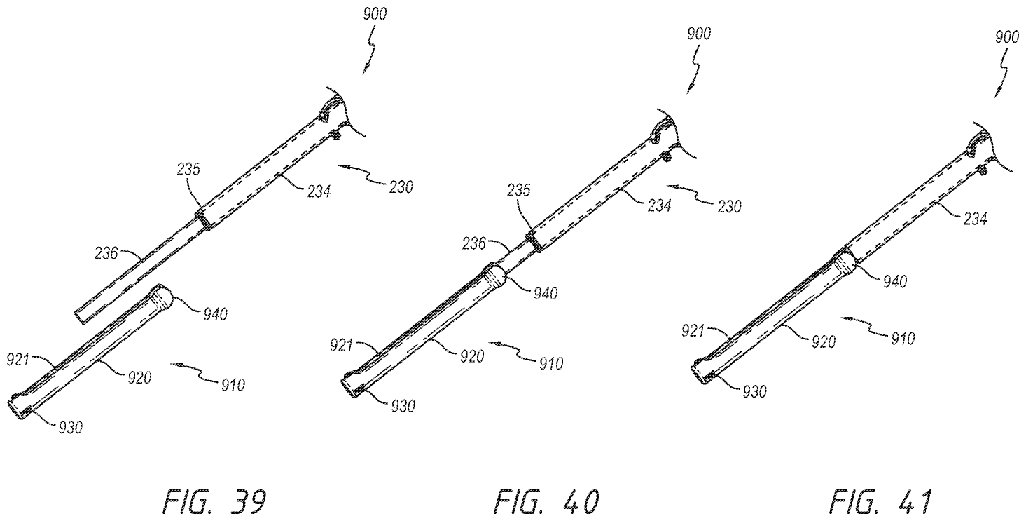

With reference to FIGS. 39-41, a cannula reducer 910 can include a latching mechanism that engages with a lip 235 extending from the surgical instrument 230. As illustrated, the cannula reducer 910 can include a proximal end 940 configured to engage with a lip 235 defined on the distal end of the instrument sleeve 234. In the depicted example, the proximal end 940 defines a circumferentially extending portion that can extend over, around or otherwise engage with the lip 235. The engagement of the proximal end 940 with the lip 235 can limit or prevent axial movement of the cannula reducer 910 relative to a portion of the surgical instrument 230.

In the depicted example, the proximal end 940 can move to engage and disengage from the lip 235. For example, the proximal end 940 can snap, deform, or otherwise resiliently engage with the lip 235 to retain the cannula reducer 910 relative to the surgical instrument 230. The proximal end 940 may be deformed to disengage the lip 235 and release the cannula reducer 910 from the surgical instrument 230.

In some embodiments the cannula reducer can be inserted, coupled, or docked with the manipulator and/or inserted into the cannula separate from the surgical instrument. Further, the cannula reducer can be positioned within the cannula by an inserter that is releasably attached to the cannula reducer.

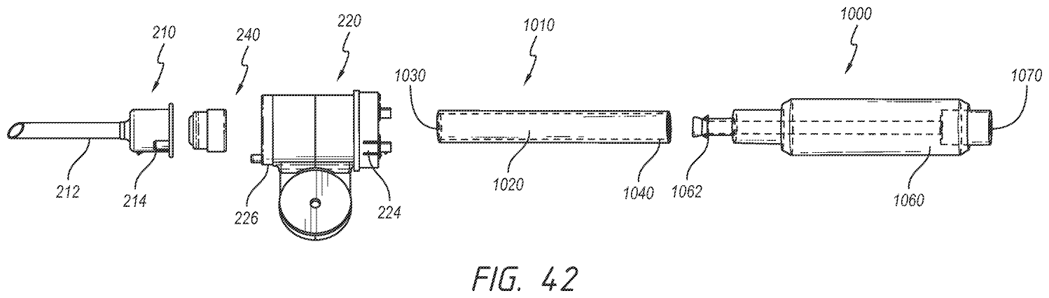

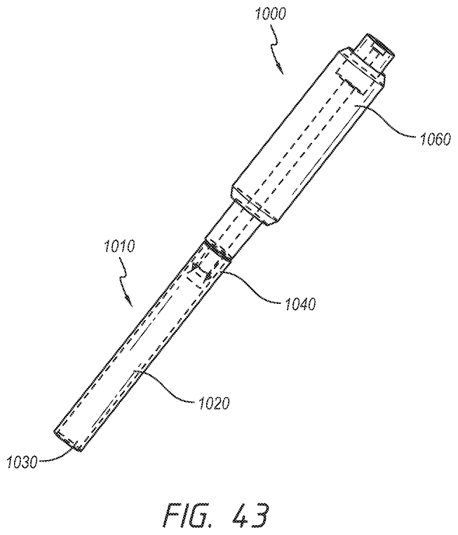

For example, with reference to FIGS. 42 and 43, the cannula reducer 1010 can be introduced into the manipulator 220 and/or the cannula 210 using an inserter 1060. The inserter 1060 can be configured to load the reducer 1010 into the cannula 210 through the passage of the manipulator 220 after the manipulator is docked to the cannula 210. During insertion, a proximal end 1040 of the cannula reducer 1010 can be coupled or attached to the inserter 1060. For example, the proximal end 1040 of the cannula reducer 1010 can attach engage, or latch to an engagement feature 1062 of the inserter 1060. In the depicted example, the proximal end 1040 can include a groove, recess, or other complimentary feature to engage with the engagement feature 1062. The engagement of the proximal end 1040 with the engagement feature 1062 can allow the cannula reducer 1010 to be axially advanced by the inserter 1060 into the manipulator 220 and the cannula 210.

In some embodiments, the engagement feature 1062 can move to engage and disengage from the proximal end 1040 of the cannula reducer 1010. For example, the engagement feature 1062 may expand radially outward to engage against the proximal end 1040 of the cannula reducer 1010 and retain the cannula reducer 1010 relative to the inserter 1060 to allow the cannula reducer 1010 to be positioned by the inserter 1060 as an assembled unit 1000.

The engagement feature 1062 may contract radially inward to disengage from the proximal end 1040 to release the cannula reducer 1010 from the inserter 1060, allowing the cannula reducer 1010 to be installed within the cannula 210. Optionally, the movement of the engagement feature 1062 can be controlled by depressing a release button 1070. For example, by actuating the release button 1070, the cannula reducer 1010 can be released from the inserter 1060 and installed within the cannula 210.

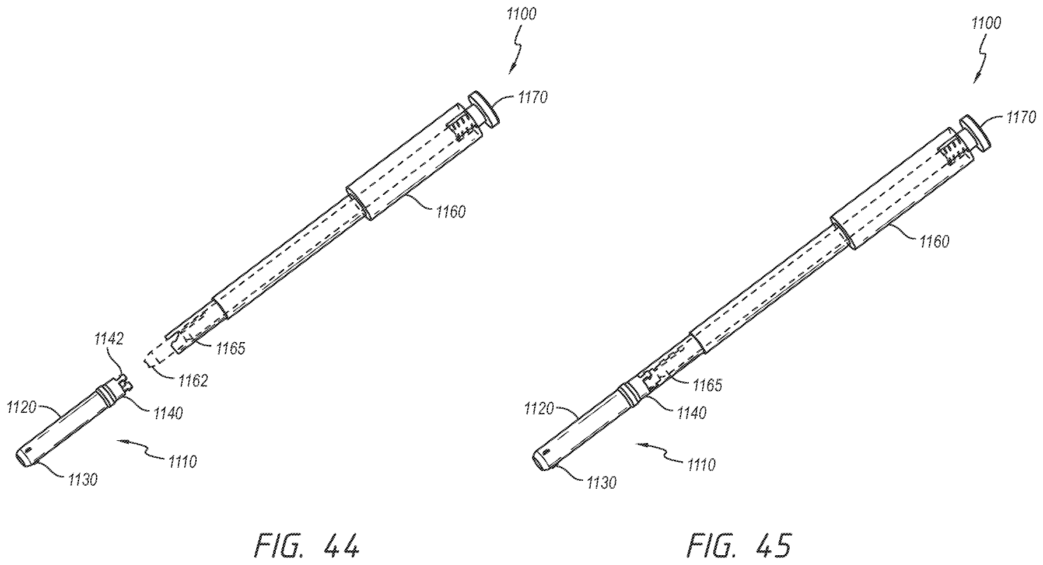

With reference to FIGS. 44 and 45, a cannula reducer 1110 can include latching features 1142 that releasably engage with the inserter 1160. During insertion, the latching features 1142 can engage with mating features 1165 defined in the surface of the inserter 1160. In the depicted example, the latching features 1142 can include axially and circumferentially extending hooked portions that engage with the mating features 1165. The engagement of the latching features 1142 can prevent axial and/or rotational movement of the cannula reducer 1110 relative to the inserter 1160 during insertion of the cannula reducer 1110.

In the depicted example, the latching features 1142 and/or the mating features 1165 can move, deflect, or deform to engage and disengage the cannula reducer 1110 from the inserter 1160. For example, the mating features 1165 may move circumferentially inward to engage the latching features 1142 and retain the cannula reducer 1110 together with the inserter 1160 as an assembled unit 1100 during insertion. The mating features 1165 can be moved circumferentially outward to disengage the latching features 1145 from the inserter 1060, releasing and positioning the cannula reducer 1110 within the cannula. In some embodiments, the engagement feature 1162 can distally urge or advance the cannula reducer 1110 relative to the inserter 1160, releasing the latching features 1142 from the mating features 1165 and releasing and positioning the cannula reducer 1110. Optionally, the movement of the engagement feature 1162 can be controlled by depressing a release button 1170.

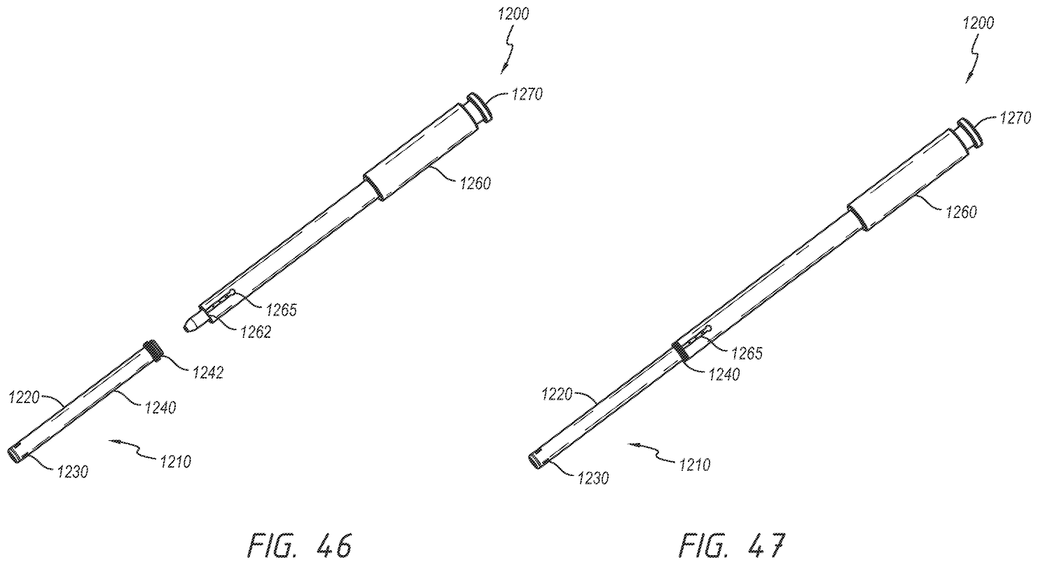

With reference to FIGS. 46 and 47, a cannula reducer 1210 can include a lip 1242 that can releasably engage with engagement features 1262 of the inserter 1260. During insertion, the engagement features 1262 of the inserter 1260 can engage with the lip 1242 defined on the surface at the distal end 1240 of the cannula reducer 1210. In the depicted example, the engagement features 1262 can include a circumferentially extending portion that can extend over, around or otherwise engage with the lip 1242. The engagement of the engagement features 1262 with the lip 1242 can prevent axial movement of the cannula reducer 1210 relative to the inserter 1260 during insertion of the cannula reducer 1210.

In the depicted example, the engagement features 1262 can move, deflect, or deform to engage and disengage the cannula reducer 1210 from the inserter 1260. For example, the engagement features 1262 snap, deform, or otherwise resiliently engage with the lip 1242 and retain the cannula reducer 1210 with the inserter 1260 as an assembled unit 1200 during insertion. The engagement features 1262 can be deformed to disengage the lip 1242 from the inserter 1260, releasing and positioning the cannula reducer 1210. In some embodiments, the engagement feature 1262 can distally urge or advance the cannula reducer 1210 relative to the inserter 1260, releasing the engagement features 1262 from the lip 1242 and releasing and positioning the cannula reducer 1210. Optionally, the movement of the engagement feature 1262 can be controlled by depressing a release button 1270.

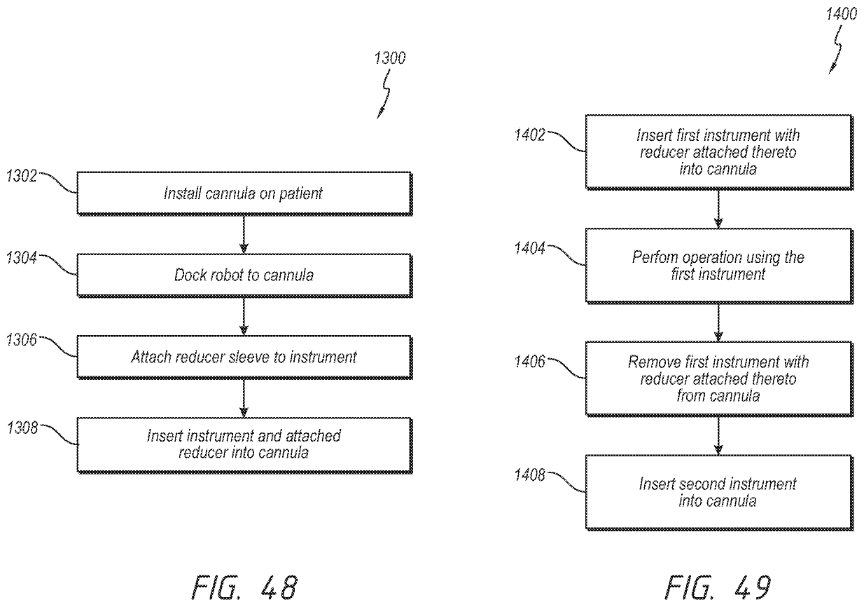

With reference to FIG. 48, a method 1300 of using a cannula reducer is described herein. In step 1302, a cannula is installed on the patient. The cannula may be inserted into a patient at a desired entry opening for a procedure. The cannula may displace or dissect soft tissue to allow the cannula to be inserted into the patient cavity to provide access to a surgical site. Optionally, an obturator may be inserted through the cannula during an initial insertion to provide a dilating or blunt tip that facilitates insertion of the cannula through an incision or other opening on the patient.

In step 1304, a robotic arm and/or manipulator is moved to the position of the cannula. The manipulator can be docked or otherwise attached to the cannula.

In step 1306, as described herein, a cannula reducer can be attached to a surgical instrument that is smaller than intended for the cannula installed within the patient. The cannula reducer and the surgical instrument can be attached together to form an assembled unit. In some embodiments, a proximal end of the cannula reducer can be coupled or attached to the surgical instrument. For example, the proximal end of the cannula reducer can attach to various features of the surgical instrument extending along the length of the instrument shaft. In the depicted example, the proximal end of the cannula reducer can attach to various features on an outer portion of the surgical instrument.

In step 1308, the assembled unit can be inserted, coupled, or docked with the manipulator and/or inserted into the cannula. When in a docked configuration, the instrument, the reducer, and cannula may be coaxially aligned along an insertion axis such that the instrument shaft can extend through the cannula. The lumen of the cannula shaft can support and coaxially align the cannula reducer and in turn, the shaft of the surgical instrument within the cannula shaft.

In some embodiments, the cannula reducer and/or the surgical instrument can be configured such that when the cannula reducer and the surgical instrument are inserted into or docked with the cannula, the proximal end of the cannula reducer can be positioned distal to a sealing layer disposed within the seal unit.

With reference to FIG. 49, a method 1400 of using a cannula reducer and exchanging surgical instruments is described herein. As described in step 1308, in step 1402, a cannula reducer and a surgical instrument can be inserted, coupled, or docked with a manipulator and/or inserted into the cannula. In step 1404, a surgical task or procedure using the surgical instrument is performed. The surgical task or procedure can include laparoscopic, urological, endoscopic, percutaneous, orthopedic, and/or or other medical or minimally invasive procedures in which a surgical instrument is introduced to the site through the cannula.

After the surgical task or procedure is completed, in step 1406, the surgical instrument and the cannula reducer can be removed as an assembled unit. Advantageously, by removing the cannula reducer and the surgical instrument as a unit, surgical instruments can be rapidly exchanged in and out of the cannula. Optionally, upon removal of the assembled unit from the cannula and/or the manipulator, the cannula reducer can be removed from the surgical instrument.

Upon removal of the first surgical instrument and cannula reducer, in step 1408, a second surgical instrument can be introduced into a cannula for a subsequent surgical task or procedure. The second surgical instrument may be inserted with a cannula reducer or without a cannula reducer.

Although embodiments are described herein with respect to cannulas, the reducers described herein may be applied to other medical devices. For example, any of the reducers disclosed herein may be applied to other medical devices besides cannulas or surgical instruments, including, for example, other access devices.

### 3. Implementing Systems and Terminology

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

The previous description of the disclosed implementations is provided to enable any person skilled in the art to make or use the present invention. Various modifications to these implementations will be readily apparent to those skilled in the art, and the generic principles defined herein may be applied to other implementations without departing from the scope of the invention. For example, it will be appreciated that one of ordinary skill in the art will be able to employ a number corresponding alternative and equivalent structural details, such as equivalent ways of fastening, mounting, coupling, or engaging tool components, equivalent mechanisms for producing particular actuation motions, and equivalent mechanisms for delivering electrical energy. Thus, the present invention is not intended to be limited to the implementations shown herein but is to be accorded the widest scope consistent with the principles and novel features disclosed herein.

What is claimed is:

1. A cannula reducer comprising:
a sleeve having a proximal end and a distal end, the sleeve being configured to be inserted into a cannula and having an interior passage configured to receive a surgical instrument, wherein the proximal end of the sleeve is configured to attach to the surgical instrument.

2. The cannula reducer of claim 1, wherein the proximal end of the sleeve is configured to attach to the surgical instrument along a shaft of the surgical instrument.

3. The cannula reducer of claim 1, wherein, in a docked configuration where the sleeve and the surgical instrument are docked to the cannula, the proximal end of the sleeve is configured to attach to the surgical instrument at a location distal to a sealing layer coupled to the cannula.

4. The cannula reducer of claim 1, wherein the proximal end of the sleeve comprises a latch member configured to engage with a housing of the surgical instrument, wherein the latch member is coupled to the sleeve via a flexural portion, wherein the latch member is integrally formed with the sleeve, wherein the sleeve further comprises an actuation portion coupled to the latch member, and wherein movement of the actuation portion moves the latch member and disengages the sleeve from the surgical instrument.

5. The cannula reducer of claim 1, wherein the sleeve comprises a tubular body extending from the proximal end to the distal end, wherein the interior passage is a lumen extending through the tubular body.

6. The cannula reducer of claim 1, wherein the sleeve comprises a lateral channel extending along a portion of the sleeve between the proximal end and the distal end, wherein the lateral channel is sized to permit passage of a shaft of the surgical instrument during lateral attachment of the sleeve to the surgical instrument.

7. The cannula reducer of claim 1, wherein the distal end of the sleeve comprises one or more radial extensions configured to engage with an inner surface of the cannula, and one or more radial gaps angularly offset from the one or more radial extensions and configured to permit fluid flow between an outer surface of the sleeve and the inner surface of the cannula.

8. A cannula reducer comprising:
a hollow body having a proximal end configured to releasably couple with an engagement feature of a surgical instrument,
wherein the cannula reducer and the surgical instrument are configured to be inserted into a cannula as an assembled unit.

9. The cannula reducer of claim 8, wherein, in a docked configuration where the hollow body and the surgical instrument are docked to the cannula, the proximal end of the hollow body is configured to attach to the surgical instrument at a location distal to a sealing layer coupled to the cannula.

10. The cannula reducer of claim 8, wherein the proximal end comprises a latch member configured to engage with the engagement feature of the surgical instrument, wherein the latch member is coupled to the hollow body via a flexural portion.

11. The cannula reducer of claim 10, further comprising an actuation portion coupled to the latch member, wherein movement of the actuation portion moves the latch member.

12. The cannula reducer of claim 8, the hollow body further defining a lumen configured to receive the surgical instrument, wherein the hollow body is tubular.

13. The cannula reducer of claim 8, further defining a lateral channel extending along a portion of the hollow body between the proximal end and a distal end, wherein the lateral channel is sized to permit passage of a shaft of the surgical instrument during lateral attachment of the hollow body to the surgical instrument.

14. A surgical system comprising:
a cannula;
a surgical instrument comprising:
a base configured to mount to a robotic arm; and
a shaft extending through the base and configured to extend through the cannula; and
a reducer sleeve configured to attach to the base of the surgical instrument to permit the surgical instrument and the reducer sleeve to be inserted into the cannula as an assembled unit.

15. The surgical system of claim 14, wherein a proximal end of the reducer sleeve attaches to the surgical instrument.

16. The surgical system of claim 14, wherein the shaft of the surgical instrument is configured to advance or retract relative to the reducer sleeve and the base of the surgical instrument.

17. The surgical system of claim 14, wherein the base of the surgical instrument includes an instrument sleeve, the shaft of the surgical instrument extends through the instrument sleeve, and the reducer sleeve is configured to attach to the instrument sleeve, wherein the instrument sleeve is configured to receive the reducer sleeve.

18. The surgical system of claim 17, wherein the shaft of the surgical instrument is configured to advance or retract relative to the reducer sleeve and the instrument sleeve.

19. The surgical system of claim 17, wherein the instrument sleeve defines an engagement feature configured to engage with the reducer sleeve, and wherein the engagement feature comprises a lip extending radially from a surface of the instrument sleeve and the reducer sleeve is configured to engage the lip.

20. The surgical system of claim 14, wherein the robotic arm comprises a robotic manipulator having a first end configured to attach to the cannula, a second end configured to attach to the base of the surgical instrument, and at least one actuator configured to actuate the surgical instrument, and the robotic arm includes at least one actuator configured to move the robotic manipulator relative to a patient.
